SEQUENCE LISTING (11 sequences)

SEQ ID NO: 1 - DNA, 1153 bp, Chlorella zofingiensis
  CDS (209)..(1147): beta carotene ketolase
ggatccaagctttgcaattcatacagaagtgagaaaaatggcttctatgatatcctcttcagctgtgactacagtcagccgtgcttctacggtgcaatcggccgcggtggctccattcggcggcctcaaatccatgactggattcccagttaagaaggtcaacactgacattacttccattacaagcaatggtggaagagtaaagtgcatggcgccagatgtgacacatgtgcagccacgtgtacagtccccggctggccccgatgatgaggatgacgcgttaagcttgtggaaagcccaatatcctatgccggaggagaagggtacagtatccaaacctcaagccgcactcaaatacaggccaccacgcagtgactggaagggtgtatcaattgcatgcactgtcatcaccctatggacagctgtcttttaccatggctgctggcaaatcaaactcacaggccctgataagtcagcctggtgggacgttgttgcaacgtttctggcactggagttcctcaacactgggcttttcatcaccacgcatgatgccatgcatgggactattgccatcaggaaccgtcgtttgaatgacctacttggcaatatagccatcagcctatatgcctggtttgactatgacatgctgcacaagaagcactgggagcatcacaacttcactgggttaccacataaagacccagacttccatcgaggcgatcctgcgctacataagtggtttggcaggtttatgtgggagtatgcaacaccactccagtttgccaagatcttcgcataccccttcttcctacaatccttacgggtgcaataccccaatttatgcgtctttctggcggctgcacccctggtcagtgcgttccgattgttctattttggcacctatttaccccacctcccctccaatgctcaggagacaatgccctgggagaaatctcacagtgctgatgaccctcggccgctgtcattcttgaaatgttatcactttgattatcactgggagcatcacaggtggccttatgccccttggtgggagttacccgtgtgtaagcgcatcacaaagacactggatgctgcagttccaggagtacagtcagacggcacgaagaagagtcagttggtgaactaagaattc

SEQ ID NO: 2 - protein, 312 aa, Chlorella zofingiensis
MAPDVTHVQPRVQSPAGPDDEDDALSLWKAQYPMPEEKGTVSKPQAALKYRPPRSDWKGVSIACTVITLWTAVFYHGCWQIKLTGPDKSAWWDVVATFLALEFLNTGLFITTHDAMHGTIAIRNRRLNDLLGNIAISLYAWFDYDMLHKKHWEHHNFTGLPHKDPDFHRGDPALHKWFGRFMWEYATPLQFAKIFAYPFFLQSLRVQYPNLCVFLAAAPLVSAFRLFYFGTYLPHLPSNAQETMPWEKSHSADDPRPLSFLKCYHFDYHWEHHRWPYAPWWELPVCKRITKTLDAAVPGVQSDGTKKSQLVN

SEQ ID NO: 3 - DNA, 24 bp, Artificial sequence
  synthetic sequence genetically recombined sequence
gcgcatatggctcccaggcggcaa

SEQ ID NO: 4 - DNA, 26 bp, Artificial sequence
  synthetic sequence genetically recombined sequence
cggtcgacttactccactactgctcc

SEQ ID NO: 5 - DNA, 1010 bp, Scenedesmus vacuolatus
  CDS (7)..(1002)
gcgcatatggctcccaggcggcaatcaacgctgccgcagcagaccaaagctggctctccaaccagtggctcagatgctgccatccctgagcccgatgtcatcgacgtgtggaaagcgcaataccctctgccggatgaaaatgtagcagggagcatgaatgaggtcaagcagttgtacaggccacctcgcaatgatgtgaagggcataagcattgccttgggcctgattgcagcctggtgcgtgctgttttaccacggctgctggcagatccagctgtctggcagtcagcgctcctggtggattgacattgctggcacatttattttgttggagttcgtcaacacaggccttttcatcaccacgcacgatgccatgcatggcactgtttgttacaggaaccgcaagctgaacgatctgttgggtcgtatagccatcacactgtacgcctggtttgactacgacatgcttcacaggaagcactgggagcatcacaactacacaggacagaagggcaaagaccctgacttccacaggggcaaccctgcactgccagtgtggtatgccaggttcatgtgggaatactccacccccttgcagtttgccaaaatcatcctggtgagtcaggtgctgcaagccctgggagtgccctacaacaacctgtgtgtctacatggctgctgcgcccctggtggccgccttcaggctgttctattttggcacctacctgccgcacttgccccccaacgcccaggaggtgatggtgtggcagaagagtcactctagtgatgctccctcctggctgtctttcctcaagtgttaccactttgattatcattgggaacaccacagatggccatatgctccctggtgggagttgccgaaggcgaagaaaattacacaacaaactcagcatcaccaacaaaccaagcagcagcagcccatgcagcaggcaaaagcgcaggttgtctcccagctggcccctgcaggagcagtagtggagtaagtcgaccg

SEQ ID NO: 6 - protein, 331 aa, Scenedesmus vacuolatus
MAPRRQSTLPQQTKAGSPTSGSDAAIPEPDVIDVWKAQYPLPDENVAGSMNEVKQLYRPPRNDVKGISIALGLIAAWCVLFYHGCWQIQLSGSQRSWWIDIAGTFILLEFVNTGLFITTHDAMHGTVCYRNRKLNDLLGRIAITLYAWFDYDMLHRKHWEHHNYTGQKGKDPDFHRGNPALPVWYARFMWEYSTPLQFAKIILVSQVLQALGVPYNNLCVYMAAAPLVAAFRLFYFGTYLPHLPPNAQEVMVWQKSHSSDAPSWLSFLKCYHFDYHWEHHRWPYAPWWELPKAKKITQQTQHHQQTKQQQPMQQAKAQVVSQLAPAGAVVE

SEQ ID NO: 7 - DNA, 5905 bp, Artificial sequence
  synthetic sequence genetically recombined sequence
gattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtccatggaacgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtaacgcgtggtgattttgtgccgagctgccggtcggggagctgttggctggctggtggcaggatatattgtggtgtaaacaaattgacgcttagacaacttaataacacattgcggacgtctttaatgtactgaattaacatccgtttgatacttgtctaaaattggctgatttcgagtgcatctatgcataaaaacaatctaatgacaattattaccaagcag

SEQ ID NO: 8 - DNA, 5027 bp, Artificial sequence
  synthetic sequence genetically recombined sequence
ctgatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctcgacatgtctccggagaggagaccagttgagattaggccagctacagcagccgatatggccgcggtttgtgacatcgttaaccattacattgagacgtctacagtgaactttaggacagagccacaaacaccacaagagtggattgatgacctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatctacattgtacacacatttgcttaagtctatggaggcgcaaggttttaagtctgtggttgctgttataggccttccaaacgatccatctgttaggttgcatgaggctttgggatacacagcgcggggtacattgcgcgcggctggatacaagcatggtggatggcatgatgttggtttttggcaaagggattttgagttgccagctcctccaaggccagttaggccagttacccagatctgagtcgatcgaccgatcttgctgcgttcggatattttcgtggagttcccgccacagacccggatgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggcctgagtcgttgtaaaacgacggccagtgaattatccaactttgtataataaagttgccatgattacgccaagcttgcatgccgtcgaccagatctgatatctgcggccgcctcgagcatatgctagaggatccccgggtaccccactttgtacaagaaagctgggtggtaccgggccccccctcgagtttaaaccactccgccctacactcgtatatatatgcctaaacctgccccgttcctcatatgtgatattattatttcattattaggtataagatagtaaacgataaggaaagacaatttattgagaaagccatgctaaaatatagatagatataccttagcaggtgtttattttacaacataacataacatagtagctagccagcaggcaggctaaaacatagtatagtctatctgcaggggaattcttagttcaccaactgactcttcttcgtgccgtctgactgtactcctggaactgcagcatccagtgtctttgtgatgcgcttacacacgggtaactcccaccaaggggcataaggccacctgtgatgctcccagtgataatcaaagtgataacatttcaagaatgacagcggccgagggtcatcagcactgtgagatttctcccagggcattgtctcctgagcattggaggggaggtggggtaaataggtgccaaaatagaacaatcggaacgcactgaccaggggtgcagccgccagaaagacgcataaattggggtattgcacccgtaaggattgtaggaagaaggggtatgcgaagatcttggcaaactggagtggtgttgcatactcccacataaacctgccaaaccacttatgtagcgcaggatcgcctcgatggaagtctgggtctttatgtggtaacccagtgaagttgtgatgctcccagtgcttcttgtgcagcatgtcatagtcaaaccaggcatataggctgatggctatattgccaagtaggtcattcaaacgacggttcctgatggcaatagtcccatgcatggcatcatgcgtggtgatgaaaagcccagtgttgaggaactccagtgccagaaacgttgcaacaacgtcccaccaggctgacttatcagggcctgtgagtttgatttgccagcagccatggtaaaagacagctgtccatagggtgatgacagtgcatgcaattgatacacccttccagtcactgcgtggtggcctgtatttgagtgcggcttgaggtttggatactgtacccttctcctccggcataggatattgggctttccacaagcttaacgcgtcatcctcatcatcggggccagccggggactgtacacgtggctgcacatgtgtcacatctggcgccatgcactttactcttccaccattgcttgtaatggaagtaatgtcagtgttgaccttcttaactgggaatccagtcatggatttgaggccgccgaatggagccaccgcggccgattgcaccgtagaagcacggctgactgtagtcacagctgaagaggatatcatagaagccatttttctcacttctgtatgaattgcaaagcttggatcctgaaatcgaaattcagagttttgatagtgagagcaaagagggacggacttatgaggatttcgagtatttcaagagatggtacttgttgatcggacggctacgatgatctcgatttggttaatccagtatctcgcggtgtatggagttatggtagggttaatggtcaatttcatctaacggtagagaatgatgtaattagataagaatcttgagatactggtttagattggatgagtgtagggtccatcttatcttgataagtggatggtttttagagacacagtgaatattagccaatcgaagttccatatcaccatcatcatctgtataattttgtttttttggaagataataatgattgaaattttggtagattttatttttcattatttaccttgtatgttgagtggtcttcaaattattgaacgtgacagattcacaagaaagtagattttttataaatgaaattttacttattttaaaggtatctctatttaatttcttttgtttatggttgtctgtcagcatttgacttgcagtttcatgctcatagtcatatacgttattctaggcttttttgaatatcttattacttttttcgtaatacaattttataattttatcaaagttatacaactataactaaaattagggttttctacaaaacaaaaaaatcttctaattttttttgttgtagccagtttactcgtaagttacaaaaaaatacaaatgaacccacatgtattatgcgtttaactaggattaccatgtactttcatgtactcaattcaccctatactctttttttttttttttctagttccacccaatctataaaattctgtccatttgaccaaattcaattaatttctgtaattgcgatttaaaattaatattacatgttcactatttctcgatttgagggaacccgagtttaaatatgataaaaatgttgacccatcactacaaatatgttatagtttatacttaatagtggtgtttttggggataattgatgaattaagtaaacatgattcttcttatgaagttgattgagtgattattgtatgtaaacctatgtgattgatgttattggttgattgagtgattattgtattagtatgtaagcaaagatgattgttcttatgaggtaatttgttactcattcatccttttgcatatgagaaattgtgttagcgtacgcaaaacaatagagaacataaaagatatgtgtatttatttaaggtgacttttgttaatgatattgtagtatctatacatttatatataacttgttgaatttgagtataagctatcattttctaattattggttgaatttatactttaaaattaggtgcaaatatattagatgagtagtccattgatctaaatatttcgcataataattttaagtactaaaatattgttgacatattttataatttttaaactttaaattttttatataagcttttgttgacacttaattcgtttaaatttggttaatactgtaataatatgtactttgttgcctattagagagacaccctctaaatcgtccagagactcttctctttgcagctaaagtctcccatttcataaaccctagccaaaactggtgagcgattcttctccggcaatttgattaaccttctagttcgtaacgagtttcattgttgcttccttcacttttggaactcgtatgttttgaagatcaaatgatttttctggtctgtgtttctttcatttggtttgggcctatcgaaatcgtttatgcacacaacctgttcgacgtagtgcgtagctgagattcttctctataaaaaggtgtattctttgctattgttggaaggtatcattgtttagtaatgggtaacaaagtgatgatgttcaagtggtcaaagaacatcacgccttcacaggttatcaaactgatgcgagctgaaaaagatgtggagaagtctatggcagtttttgattccgcaacggcggaatacgcaaatgggtacgtacatgatcagagttcttttggttatgagatcagatctggtcgacggcatgcaagcttggcgtaatcatggagcctgcttttttgtacaaacttgccatgattacgccaagcttgcatgccgtcgaccagatctgatatctgcggccgcctcgagcatatgctagaggatccccgggtacccaacttttctatacaaagttggatggcatgcaagcttggcgtaatcatggtcatagctgtttcctactagatct

SEQ ID NO: 9 - DNA, 4157 bp, Artificial sequence
  synthetic sequence genetically recombined sequence
ctgatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctcgacatgtctccggagaggagaccagttgagattaggccagctacagcagccgatatggccgcggtttgtgacatcgttaaccattacattgagacgtctacagtgaactttaggacagagccacaaacaccacaagagtggattgatgacctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatctacattgtacacacatttgcttaagtctatggaggcgcaaggttttaagtctgtggttgctgttataggccttccaaacgatccatctgttaggttgcatgaggctttgggatacacagcgcggggtacattgcgcgcggctggatacaagcatggtggatggcatgatgttggtttttggcaaagggattttgagttgccagctcctccaaggccagttaggccagttacccagatctgagtcgatcgaccgatcttgctgcgttcggatattttcgtggagttcccgccacagacccggatgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggcctgagtcgttgtaaaacgacggccagtgaattatccaactttgtataataaagttgccatgattacgccaagcttgcatgccgtcgaccagatctgatatctgcggccgcctcgagcatatgctagaggatccccgggtaccccactttgtacaagaaagctgggtggtaccgggccccccctcgagtttaaaccactccgccctacactcgtatatatatgcctaaacctgccccgttcctcatatgtgatattattatttcattattaggtataagatagtaaacgataaggaaagacaatttattgagaaagccatgctaaaatatagatagatataccttagcaggtgtttattttacaacataacataacatagtagctagccagcaggcaggctaaaacatagtatagtctatctgcatgctcgagcggccgccagtgtgatggatatctgcagaattcgcccttcggtcgacttactccactactgctcctgcaggggccagctgggagacaacctgcgcttttgcctgctgcatgggctgctgctgcttggtttgttggtgatgctgagtttgttgtgtaattttcttcgccttcggcaactcccaccagggagcatatggccatctgtggtgttcccaatgataatcaaagtggtaacacttgaggaaagacagccaggagggagcatcactagagtgactcttctgccacaccatcacctcctgggcgttggggggcaagtgcggcaggtaggtgccaaaatagaacagcctgaaggcggccaccaggggcgcagcagccatgtagacacacaggttgttgtagggcactcccagggcttgcagcacctgactcaccaggatgattttggcaaactgcaagggggtggagtattcccacatgaacctggcataccacactggcagtgcagggttgcccctgtggaagtcagggtctttgcccttctgtcctgtgtagttgtgatgctcccagtgcttcctgtgaagcatgtcgtagtcaaaccaggcgtacagtgtgatggctatacgacccaacagatcgttcagcttgcggttcctgtaacaaacagtgccatgcatggcatcgtgcgtggtgatgaaaaggcctgtgttgacgaactccaacaaaataaatgtgccagcaatgtcaatccaccaggagcgctgactgccagacagctggatctgccagcagccgtggtaaaacagcacgcaccaggctgcaatcaggcccaaggcaatgcttatgcccttcacatcattgcgaggtggcctgtacaactgcttgacctcattcatgctccctgctacattttcatccggcagagggtattgcgctttccacacgtcgatgacatcgggctcagggatggcagcatctgagccactggttggagagccagctttggtctgctgcggcagcgttgattgccgcctgggagccatgcactttactcttccaccattgcttgtaatggaagtaatgtcagtgttgaccttcttaactgggaatccagtcatggatttgaggccgccgaatggagccaccgcggccgattgcaccgtagaagcacggctgactgtagtcacagctgaagaggatatcatagaagccatttttctcacttctgtatgaattgcaaagctatctcgaagctagtgaacaagaaaaatgtacatacactgactactaatttatagccatcattactgctgatttggcaaatggcgtaatctcaaaccaaatcacgaggggtaataaatgttagccacataaattggtaagatcaaaggacgatattcagacatcttaagcatcgtggtgtgaggcgataattgttttgggctttttggctcttgtttatggcaagaaaatccaatttgacacatgctggctaattttcaaaaagcataaaggattagtcagggattctgaaactaggagtgcaattttacacacagaaaaatcacataatcgtaacctataatcacgatgtgattcaatgccatgattatatcttcaaactgaaatcatagagctcattaaattgttgtcttttgttatatattaatggtacataatgccctttaaatgcttttgtactttgcgaaattttgaaataaacaaaatacattgtttctgtatgcacatggaaagtactaataaacgaaaaagaaagtttctctgcaaaaaacaaaaaaaaatatatttagggatagacagaaatcattgtttaggaaagttgctctttattatctctactacttctccacgagaattaggcgccatgcaaatgacaccgatgctagaaacttcggcagtgtactattgtctggccgttttctgattatcgctcagtttgccaactactttggtcctttaacggtcgagatctgctaccatgcgataaatatcttgacagcgttttggaaagcgcgattgagaggaaacgtccaaactatttgtttattgggcttataattgactaaaaatgggccttgttaacagaatctcttgtattttttttcttttgaaatttacatcacttttccataacaaacgaaatgcaacaaactgtaaaaaaactttgtgttagatggagaatttacagccattcgattggtttcaaaatatgtcaagagtctcaatgatcggacggttgcaaagaggacacgagcctctgcagcgcaagggcgaattccagcacactggcggccgttactagtggatccgagctcggtaccaagcttggcgtaatcatggagcctgcttttttgtacaaacttgccatgattacgccaagcttgcatgccgtcgaccagatctgatatctgcggccgcctcgagcatatgctagaggatccccgggtacccaacttttctatacaaagttggatggcatgcaagcttggcgtaatcatggtcatagctgtttcctactagatct

SEQ ID NO: 10 - DNA, 960 bp, Brassica napus
ggtaccaggctcgtgtcctctttgcaaccgtccgatcattgagatcttagacatattttgaaaccaatcgaatggctgtaaattctcgatctaacacagtttatacattcgtttgttatgggaagatgatgtaaatttcaaaaaaatacaagatattctgttcagaaggcccattaagtcaattttaagcccaataaaccaatagtttggacgtttcctctctatcgcgcttttccgaaacgccagaaaaacgctgtcaagatatttatcgcatggtagcagatctcgaccgttaaaggaccaaagtagttggcaaactgagcgataatcagaaaacagccagacaatagtacaccgccgaagtttctagcatcgtgtcatttgcatggcgcctaattctcgtcgagaactagagataataaagagcaactttcctaaacaataatttctgtctatccctaaatatttttttttgttctttgcagagaaactttctttttcatttattagtactttccatgtgcacagagaaacaatgtattttgtttacttcaaaatttcacaaagtacaaaagcatttaaaggacattatgtgacacacgtcatacaatttaatgagctctatgactttcagtttgaagatctaattatagcattgaatcacatcgtaattacagattatgattatctgtgtgtaaaattgtactcctagtttcagaatcccttactaatcaatcctttctacattttgaaaaattagtcagcatgtgtcaaattggatttcttgccataaacaagagccaaaaaaaacccaaaacaatgatcgcctcacaccacgatgcttaagatgtctgtctgattatcgtgcctttgatcttaccaatttatgtggctaacatttattacccctcgtgatttggtttgagattacgccatttgccaaataagcagtaaaagctt

SEQ ID NO: 11 - DNA, 1043 bp, Brassica napus
ggtaccaggctcgtgtcctctttgcaaccgtccgatcattgagactcttgacatattttgaaaccaatcgaatggctgtaaattctccatctaacacaaagtttttttacagtttgttgcatttcgtttgttatggaaaagtgatgtaaatttcaaaagaaaaaaaatacaagagattctgttaacaaggcccatttttagtcaattataagcccaataaacaaatagtttggacgtttcctctcaatcgcgctttccaaaacgctgtcaagatatttatcgcatggtagcagatctcgaccgttaaaggaccaaagtagttggcaaactgagcgataatcagaaaacggccagacaatagtacactgccgaagtttctagcatcggtgtcatttgcatggcgcctaattctcgtggagaagtagtagagataataaagagcaactttcctaaacaatgatttctgtctatccctaaatatatttttttttgttttttgcagagaaactttctttttcgtttattagtactttccatgtgcatacagaaacaatgtattttgtttatttcaaaatttcgcaaagtacaaaagcatttaaagggcattatgtaccattaatatataacaaaagacaacaatttaatgagctctatgatttcagtttgaagatataatcatggcattgaatcacatcgtgattataggttacgattatgtgatttttctgtgtgtaaaattgcactcctagtttcagaatccctgactaatcctttatgctttttgaaaattagccagcatgtgtcaaattggattttcttgccataaacaagagccaaaaagcccaaaacaattatcgcctcacaccacgatgcttaagatgtctgaatatcgtcctttgatcttaccaatttatgtggctaacatttattacccctcgtgatttggtttgagattacgccatttgccaaatcagcagtaatgatggctataaattagtagtcagtgtatgtacatttttcttgttcactagcttcgaaagctt

(19) United States
(12) Patent Application Publication
(10) Pub. No.: US 2010/0005541 A1
(43) Pub. Date: Jan. 7, 2010

(54) KETOLASES FOR THE PRODUCTION OF KETOCAROTENOIDS IN TAGETES

(75) Inventors: George Mather Sauer, Quedlinburg (DE); Ralf Flachmann, Quedlinburg (DE); Christel Renate Schopfer, Ludwigshafen (DE); Michael Leps, Halberstadt (DE); Hannia Bridg-Giannakopoulos, Quedlinburg (DE)

Correspondence Address: CONNOLLY BOVE LODGE & HUTZ, LLP, P O BOX 2207, WILMINGTON, DE, 19899, US

(73) Assignee: BASF Plant Science GmbH, Ludwigshafen (DE)

(21) Appl. No.: 12/514,676

(22) PCT Filed: Nov. 13, 2007
(86) PCT No.: PCT/EP07/62241

§ 371 Date: May 13, 2009

(30) Foreign Application Priority Data
Nov. 15, 2006 (EP) 06124146.9

(51) Int. Cl. C12N 15/82; C12N 9/10; C12N 15/11; A01H 5/00

(52) U.S. Cl. 800/278; 435/193; 536/23.2; 800/298

(57) ABSTRACT

New ketolases for the production of ketocarotenoids in Tagetes Abstract A ketolase having a polypeptide sequence SEQ ID NO:6, a sequence derived from SEQ ID NO:6 by substitution, insertion or deletion of aminoacids, which has an identity of at least 60% with the respective sequence SEQ. ID. NO: 6 or a functionally equivalent fragment of the sequences mentioned above and processes for production of ketocarotenoids in Tagetes.

The present invention relates to new ketolases, their use for the expression of ketocarotenoids in plants preferably for the flower-specific expression, of genes in plants of the genus Tagetes, and to a process for the preparation of keto-carotenoids by culturing the genetically modified plants.

Various biosynthetic products, such as, for example, fine chemicals, such as, inter alia, amino acids, vitamins, carotenoids, but also proteins, are prepared in cells by means of natural metabolic processes und are used in many branches of industry, including the foodstuffs, feedstuffs, cosmetic, feed, food and pharmaceutical industries. These substances, which together are described as fine chemicals/proteins, comprise, inter alia, organic acids, both proteinogenic and nonproteinogenic amino acids, nucleotides and nucleosides, lipids and fatty acids, diols, carbohydrates, aromatic compounds, vitamins, carotenoids and cofactors, and also proteins and enzymes. Their production on the large-scale in some cases takes place by means of biotechnological processes using microorganisms which were developed in order to produce and secrete large amounts of the in each case desired substance.

Carotenoids are synthesized de novo in bacteria, algae, fungi and plants. In recent years, it has increasingly been attempted also to utilize plants as production organisms for fine chemicals, in particular for vitamins and carotenoids.

A natural mixture of the carotenoids lutein, zeaxanthin and violaxanthin is extracted, for example, from the flowers of marigold plants (Tagetes plants) as “oleoresin”. This oleoresin is used both as an ingredient of food supplements and in the feed sector.

Lycopene from tomatoes is likewise used as a food supplement, while phytoene is mainly used in the cosmetic sector.

Ketocarotenoids, that is carotenoids which comprise at least one keto group, such as, for example, astaxanthin, canthaxanthin, echinenone, 3-hydroxyechinenone, 3′-hydroxyechinenone, adonirubin and adonixanthin are natural antioxidants and pigments which are produced by some algae, plants and microorganisms as secondary metabolites.

On account of their color-imparting properties, the ketocarotenoids and in particular astaxanthin are used as pigmenting aids in animal nutrition, in particular in trout, salmon and shrimp farming.

An economical biotechnological process for the production of natural, biosynthetic products and in particular carotenoids is therefore of great importance.

WO 0032788 describes some carotenoid biosynthesis genes from plants of the genus Tagetes and discloses how genetically modified plants of the genus Tagetes could be produced in order to obtain various carotenoid profiles in the petals and thus to produce certain carotenoids selectively. To this end, it is necessary to overexpress some biosynthesis genes and to suppress others.

For the overexpression of the newly found carotenoid biosynthesis genes in plants of the genus Tagetes, WO 0032788 postulates the petal-specific promoter of the ketolase from Adonis vernalis.

The ketolases used so far in the prior art do not meet all requirements which are necessary in order to effect a high expression of keto-carotenoids in Tagetes

The invention therefore relates to the use of new ketolases having a polypeptide sequence SEQ ID NO:2 or SEQ ID NO:6, a sequence derived from SEQ ID NO:2 or SEQ ID NO:6 by substitution, insertion or deletion of aminoacids, which has an identity of at least 60% with the respective sequence SEQ. ID NO:2 or SEQ ID NO:6 or a functionally equivalent fragment of the sequences mentioned above, for the expression of heterologous genes in lutein-depleted

A “ketolase” is understood as meaning a protein which has the enzymatic activity to introduce a keto group on the optionally substituted beta-ionone ring of carotenoids. This enzymatic activity is meant when the term “functionally equivalent fragment” is used in connection with ketolase polypeptides.

In particular, a ketolase is understood as meaning a protein which has the enzymatic activity to convert beta-carotene to canthaxanthin.

Examples for amino acid substitutions starting from the SEQ ID NO: 1A or SEQ ID NO:4A are listed in the following table:

| Original residue | e.g. substituted by |
|---|---|
| Ala | Ser |
| Arg | Lys |
| Asn | Gln; His |
| Asp | Glu |
| Cys | Ser |
| Gln | Asn |
| Glu | Asp |
| Gly | Pro |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

A “promoter” is understood according to the invention as meaning a nucleic acid having expression activity, that is a nucleic acid which, in functional linkage with a nucleic acid to be expressed, also described as a gene below, regulates the expression, that is the transcription and the translation of this nucleic acid or this gene.

“Transcription” is understood according to the invention as meaning the process by which, starting from a DNA matrix, a complementary RNA molecule is prepared. Proteins such as RNA polymerase, “sigma factors” and transcriptional regulator proteins are involved in this process. The RNA synthesized is then used as a matrix in the translation process, which then leads to the biosynthetically active protein.

A “functional linkage” is understood in this connection as meaning, for example, the sequential arrangement of one of the promoters according to the invention and a nucleic acid sequence to be expressed and, if appropriate, further regulative elements such as, for example, a terminator in such a way that each of the regulative elements can fulfill its function in the expression of the nucleic acid sequence. To this end, a direct linkage in the chemical sense is not imperative. Genetic control sequences, such as, for example, enhancer sequences, can also exert their function on the target sequence from positions which are further removed or even from other DNA molecules. Arrangements are preferred in which the nucleic acid sequence to be expressed or the gene to be expressed is positioned behind (i.e. at the 3′-end) of the promoter sequence according to the invention, such that both sequences are bonded covalently to one another. Preferably, the distance between the promoter sequence and the nucleic acid sequence to be expressed is in this case lower than 200 base pairs, particularly preferably less than 100 base pairs, very particularly preferably less than 50 base pairs.

“Expression activity” is understood according to the invention as meaning the amount of protein formed in a certain time by the promoter, that is the expression rate.

“Specific expression activity” is understood according to the invention as meaning the amount of protein per promoter formed in a certain time by the promoter.

In the case of a “caused expression activity” or “caused expression rate” in relation to a gene in comparison with the wild-type, in comparison with the wild-type the formation of a protein is thus caused which thus was not present in the wild-type.

In the case of an “increased expression activity” or “increased expression rate” in relation to a gene in comparison with the wild-type, in comparison with the wild-type the amount of protein formed in a certain time is thus increased.

The formation rate at which a biosynthetically active protein is prepared is a product of the rate of transcription and translation. Both rates can be influenced according to the invention and thus influence the rate of formation of products in a microorganism and plant.

“Heterologous gene expression” means according to the invention that a promoter and the functionally linked gene do not exist in such a construction in wild-types plants. Heterologous gene expression encompasses the cases the promoter or the gene or both components do not exist in the wild-type of the respective plant or where the promoter and the gene do exist in the wildtype but at different genetic locations which do not allow a functional linkage in the wildtype.

The term “wild-type” or “wild-type plant” is understood according to the invention as meaning the corresponding starting plant of the genus Tagetes.

Depending on the connection, the term “plant” can be understood as meaning the starting plant (wild-type) or a genetically modified plant according to the invention of the genus Tagetes or both.

Preferably, “wild-type” is understood as meaning the plant Tagetes erecta, in particular the plant Tagetes erecta Hybrid 50011 (WO 02012438) as well as Tagetes erecta 13819 and the derivatives produced by mutagenesis or selection as a reference organism for the increasing or causing of the expression activity or expression rate and for the increasing of the content of biosynthetic products.

Another embodiment of the invention are polynucleotide sequences encoding ketolases having a polypeptide sequence SEQ ID NO:2 or SEQ ID NO:6, a sequence derived from SEQ ID NO:2 or SEQ ID NO:6 by substitution, insertion or deletion of aminoacids, which has an identity of at least 60% with the respective sequence SEQ. ID NO:2 or SEQ ID NO:6 or a functionally equivalent fragment of the sequences mentioned above.

These polynucleotide sequences can be generated by a retranslation of the polypeptide sequence according to the genetic code. A preferred polynucleotide sequence is adapted in its codon usage to the host organism in which the polynucleotide sequence shall be expressed. All abovementioned polynucleotide sequences can be prepared in a manner known per se by chemical synthesis from the nucleotide structural units such as, for example, by fragment condensation of individual overlapping, complementary nucleic acid structural units of the double helix. The chemical synthesis of oligonucleotides can be carried out, for example, in a known manner, according to the phosphoamidite method (Voet, Voet, 2nd edition, Wiley Press New York, pp. 896-897). The addition of synthetic oligonucleotides and filling of gaps with the aid of the Klenow fragment of the DNA polymerase and ligation reactions and general cloning processes are described in Sambrook et al. (1989), Molecular cloning: A laboratory manual, Cold Spring Harbor Laboratory Press.

A preferred embodiment of the invention are polynucleotide sequences encoding ketolases wherein the polynucleotide sequences comprise sequences pursuant SEQ ID NO. 2 or SEQ ID NO:5.

A preferred embodiment of the invention are polynucleotide sequences encoding ketolases wherein this polynucleotide sequences are under the transcriptional control of a plastid-lipid associated protein promoter (PAP-promoter)

An “PAP-promoter” is understood as meaning promoters which naturally do exist preferably in plans such as cucumber, tomato and rape and regulate the gene expression of plastid-lipid associated proteins.

Preferred PAP promoters comprise
A1) the nucleic acid sequence SEQ. ID. NO:10 or 11 or
A2) a sequence derived from these sequences by substitution, insertion or deletion of nucleotides, which has an identity of at least 60% at nucleic acid level with the respective sequence SEQ. ID. NO:10 or 11 or
A3) a nucleic acid sequence which is hybridized with the nucleic acid sequence SEQ. ID. NO:10 or 11 under stringent conditions or
A4) functionally equivalent fragments of the sequences under A1), A2) or A3)

The nucleic acid sequence SEQ. ID. NO. 10 represents a promoter sequence of hypothetical plastid-lipid associated protein from Brassica napus.

The nucleic acid sequence SEQ. ID. NO. 11 represents a promoter sequence of hypothetical plastid-lipid associated protein from Brassica napus.

The invention furthermore relates to PAP promoters, comprising a sequence derived from these sequences (SEQ. ID. NO:10 or 11) by substitution, insertion or deletion of nucleotides, which has an identity of at least 60% at nucleic acid level with the respective sequence SEQ. ID. NO:10 or 11.

Further natural examples according to the invention of PAP promoters according to the invention can easily be found, for example, from various organisms whose genomic sequence is known, by identity comparisons of the nucleic acid sequences from databases containing the sequences SEQ ID NO:10 or 11 described above.

Synthetic PAP promoter sequences according to the invention can easily be found starting from the sequences SEQ ID NO: 10 or 11 by synthetic variation and mutation, for example by substitution, insertion or deletion of nucleotides.

The following definitions and conditions of the identity comparisons and hybridization conditions apply for all nucleic acids, that is all promoters and genes of the description.

The term “substitution” is to be understood as meaning the exchange of one or more nucleotides for one or more nucleotides. “Deletion” is the replacement of a nucleotide by a direct bond. Insertions are insertions of nucleotides into the nucleic acid sequence, a direct bond formally being replaced by one or more nucleotides.

Identity between two nucleic acids is understood as meaning the identity of the nucleotides over the entire nucleic acid length in each case, in particular the identity which is calculated by comparison with the aid of the Vector NTI Suite 7.1 software of Informax (USA) using the Clustal method (Higgins D G, Sharp P M. Fast and sensitive multiple sequence alignments on a microcomputer. Comput Appl. Biosci. 1989 April; 5(2):151-1) with setting of the following parameters:

Multiple alignment parameter:

| | |
|---|---|
| Gap opening penalty | 10 |
| Gap extension penalty | 10 |
| Gap separation penalty range | 8 |
| Gap separation penalty | off |
| % identity for alignment delay | 40 |
| Residue specific gaps | off |
| Hydrophilic residue gap | off |
| Transition weighing | 0 |

Pairwise alignment parameter:

| FAST algorithm | on |
|---|---|
| K-tuple size | 1 |
| Gap penalty | 3 |
| Window size | 5 |
| Number of best diagonals | 5 |

A nucleic acid sequence which has an identity of at least 60% with the sequence SEQ ID NO: N is accordingly understood as meaning a nucleic acid sequence which, in a comparison of its sequence with the sequence SEQ ID NO: N, in particular according to the above program logarithm, has an identity of at least 60% with the above parameter set.

Particularly preferred PAP promoters have, with the respective nucleic acid sequence SEQ. ID. NO. 10 or 11, an identity of at least 70%, more preferably at least 80%, at least 90%, at least 92%, at least 95%, at least 96%, at least 97%, at least 98%, particularly preferably at least 99%.

Further natural examples of PAP promoters can furthermore easily be found starting from the nucleic acid sequences described above, in particular starting from the sequences SEQ ID NO: 10 or 11, of various organisms whose genomic sequence is not known, by hybridization techniques in a manner known per se.

A further subject of the invention therefore relates to PAP promoters comprising a nucleic acid sequence which hybridizes with the nucleic acid sequence SEQ. ID. No.10 or 11 under stringent conditions. This nucleic acid sequence comprises at least 10, more preferably more than 12, 15, 30, 50 or particularly preferably more than 150 nucleotides.

“Hybridization” is understood as meaning the ability of a poly- or oligonucleotide to bind under stringent conditions to a nearly complementary sequence, while under these conditions nonspecific binding between noncomplementary partners does not happen. To this end, the sequences should preferably be complementary to 90-100%. The characteristic of complementary sequences to be able to bind specifically to one another is made use of, for example, in the Northern or Southern blot technique or in primer binding in PCR or RT-PCR.

Hybridization takes place according to the invention under stringent conditions. Such hybridization conditions are described, for example, in Sambrook, J., Fritsch, E. F., Maniatis, T., in: Molecular Cloning (A Laboratory Manual), 2nd edition, Cold Spring Harbor Laboratory Press, 1989, pages 9.31-9.57 or in Current Protocols in Molecular Biology, John Wiley & Sons, N.Y. (1989), 6.3.1-6.3.6:

Stringent hybridization conditions are in particular understood as meaning: Overnight incubation at 42° C. in a solution consisting of 50% formamide, 5×SSC (750 mM NaCl, 75 mM trisodium citrate), 50 mM sodium phosphate (ph 7.6), 5×Denhardt solution, 10% dextran sulfate and 20 g/ml of denatured, sheared salmon sperm DNA, followed by washing of the filter with 0.1×SSC at 65° C.

For promoters, a “functionally equivalent fragment” is understood as meaning fragments essentially having the same promoter activity as the starting sequence.

“Essentially identical” is understood as meaning a specific expression activity which has at least 50%, preferably 60%, more preferably 70%, more preferably 80%, more preferably 90%, particularly preferably 95%, of the specific expression activity of the starting sequence.

“Fragments” are understood as meaning subsequences of the PAP promoters described by embodiment A1), A2) or A3). Preferably, these fragments have more than 10, but more preferably more than 12, 15, 30, 50 or particularly preferably more than 150, connected nucleotides of the nucleic acid sequence SEQ. ID. NO. 9, or 12.

The use of the nucleic acid sequence SEQ. ID. NO. 10 or 11 as an PAP promoter, i.e. for the expression of genes in plants of the genus Tagetes, which are depleted in their lutein productivity, is particularly preferred.

All abovementioned PAP promoters can furthermore be prepared in a manner known per se by chemical synthesis from the nucleotide structural units such as, for example, by fragment condensation of individual overlapping, complementary nucleic acid structural units of the double helix. The chemical synthesis of oligonucleotides can be carried out, for example, in a known manner, according to the phosphoamidite method (Voet, Voet, 2nd edition, Wiley Press New York, pp. 896-897). The addition of synthetic oligonucleotides and filling of gaps with the aid of the Klenow fragment of the DNA polymerase and ligation reactions and general cloning processes are described in Sambrook et al. (1989), Molecular cloning: A laboratory manual, Cold Spring Harbor Laboratory Press.

Using the promoters according to the invention, in principle any gene, that is any nucleic acid, encoding a protein, can be expressed in plants of the genus Tagetes, in particular expressed flower-specifically, particularly preferably expressed petal-specifically.

These genes to be expressed in plants of the genus Tagetes are also called “effect genes” below.

Preferred effect genes are, for example, genes from the biosynthesis pathway of odoriferous substances and flower colors whose expression or increased expression in plants of the genus Tagetes leads to a change in the odor and/or the flower color of flowers of the plants of the genus Tagetes.

The biosynthesis of volatile odor components, especially in flowers, was in recent years studied on various model organisms such as Clarkia breweri and Antirhinum majus L. Volatile odor components are formed, for example, within the monoterpene and phenylpropane metabolism. In the first case, the component is linalool; methyleneeugenol, benzyl acetate, methyl benzoate and methyl salicate are derived from the phenylpropanes.

For the biosynthesis of linalool, (Iso)methyleigenol, benzyl acetate and methyl salicinate, preferred genes are selected from the group consisting of nucleic acids encoding a linalool synthase (LIS), nucleic acids encoding an S-adenosyl-L-Met:(iso)-eugenol O-methyltransferase (IEMT), nucleic acids encoding an acetyl-CoA-benzyl alcohol acetyltransferase and nucleic acids encoding an S-adenosyl-L-Met: salicylic acid methyltransferase (SAMT). Nucleic acid sequences and protein sequences for the enzymatic activities mentioned are described in Dudareva et al. Plant Cell 8 (1996), 1137-1148; Wang et al. Plant Physiol. 114 (1997), 213-221 and Dudareva et al. Plant J. 14 (1998) 297-304.

Particularly preferred effect genes are genes from biosynthesis pathways of biosynthetic products which can be prepared in plants of the genus Tagetes naturally, i.e. in the wild-type or by genetic modification of the wild-type, in particular can be prepared in flowers, particularly preferably can be prepared in petals.

Lutein-depleted Tagetes is a Tagetes plant, where the production of lutein has been decreased. The production of such lutein-depleted Tagetes has been disclosed in U.S. Pat. No. 6,784,351 which is herewith incorporated by reference entirely.

Preferred biosynthetic products are fine chemicals.

The term “fine chemical” is known in the specialist field and comprises compounds which are produced by an organism and are applied in various branches of industry, such as, for example, but not restricted to, the pharmaceutical industry, the agricultural industry, cosmetics, food and feed industries. These compounds comprise organic acids, such as, for example, tartaric acid, itaconic acid and diaminopimelic acid, both proteinogenic and nonproteinogenic amino acids, purine and pyrimidine bases, nucleosides and nucleotides (as described, for example, in Kuninaka, A. (1996) Nucleotides and related compounds, pp. 561-612, in Biotechnology vol. 6, Rehm et al., publ. VCH: Weinheim and the citation contained therein), lipids, saturated and unsaturated fatty acids (for example arachidonic acid), diols (for example propanediol and butanediol), carbohydrates (for example hyaluronic acid and trehalose), aromatic compounds (for example aromatic amines, vanillin and indigo), vitamins, carotenoids and cofactors (as described in Ullmann's Encyclopedia of Industrial Chemistry, vol. A27, “Vitamins”, pp. 443-613 (1996) VCH: Weinheim and the citation contained therein; and Ong, A. S., Niki, E. and Packer, L. (1995) “Nutrition, Lipids, Health and Disease” Proceedings of the UNESCO/Confederation of Scientific and Technological Associations in Malaysia and the Society for Free Radical Research—Asia, held on Sep. 1-3, 1994 in Penang, Malysia, AOCS Press (1995)), enzymes and all other chemicals described by Gutcho (1983) in Chemicals by Fermentation, Noyes Data Corporation, ISBN: 0818805086 and the references stated therein. The metabolism and the uses of certain fine chemicals are further illustrated below.

### I. Amino Acid Metabolism and Uses

The amino acids comprise the fundamental structural units of all proteins and are thus essential for the normal cell functions. The term “amino acid” is known in the specialist field. The proteinogenic amino acids, of which there are 20 types, serve as structural units for proteins, in which they are linked to one another by means of peptide bonds, whereas the nonproteinogenic amino acids (of which hundreds are known) usually do not occur in proteins (see Ullmann's Encyclopedia of Industrial Chemistry, vol. A2, pp. 57-97 VCH: Weinheim (1985)). The amino acids can be present in the D- or L-configuration, although L-amino acids are usually the only type which are found in naturally occurring proteins. Biosynthesis and degradation pathways of each of the 20 proteinogenic amino acids are well characterized both in prokaryotic and eukaryotic cells (see, for example, Stryer, L. Biochemistry, 3rd edition, pp. 578-590 (1988)). The “essential” amino acids (histidine, isoleucine, leucine, lysine, methionine, phenylalanine, threonine, tryptophan and valine), so-called as on account of the complexity of their biosynthesis they have to be assimilated with the food, are converted by means of simple biosynthesis pathways into the other 11 “nonessential” amino acids (alanine, arginine, asparagine, aspartate, cysteine, glutamate, glutamine, glycine, proline, serine and tyrosine). Higher animals possess the ability to synthesize some of these amino acids, but the essential amino acids have to be assimilated with the food in order that normal protein synthesis takes place.

Apart from their function in protein biosynthesis, these amino acids are interesting chemicals per se, and it has been discovered that many are used in various applications in the foodstuffs, feedstuffs, chemical, cosmetics, agriculture and pharmaceutical industries. Lysine is an important amino acid not only for the nutrition of humans, but also for monogastric animals, such as poultry and pigs. Glutamate is most often used as a flavor additive (monosodium glutamate, MSG) and to a great extent in the foodstuffs industry, as are also aspartate, phenylalanine, glycine and cysteine. Glycine, L-methionine and tryptophan are all used in the pharmaceutical industry. Glutamine, valine, leucine, isoleucine, histidine, arginine, proline, serine and alanine are used in the pharmaceutical industry and the cosmetics industry. Threonine, tryptophan and D-/L-methionine are widespread feedstuff additives (Leuchtenberger, W. (1996) Amino acids—technical production and use, pp. 466-502 in Rehm et al., (ed.) Biotechnology vol. 6, chapter 14a, VCH: Weinheim). It has been discovered that these amino acids are moreover suitable as precursors for the synthesis of synthetic amino acids and proteins, such as N-acetylcysteine, S-carboxymethyl-L-cysteine, (S)-5-hydroxytryptophan and other substances, described in Ullmann's Encyclopedia of Industrial Chemistry, vol. A2, pp. 57-97, VCH, Weinheim, 1985.

The biosynthesis of these natural amino acids in organisms which can produce them, for example bacteria, has been well characterized (for a general survey of bacterial amino acid biosynthesis and its regulation, see Umbarger, H. E. (1978) Ann. Rev. Biochem. 47: 533-606). Glutamate is synthesized by reductive animation of -ketoglutarate, an intermediate in the citric acid cycle. Glutamine, proline and arginine are in each case produced in succession from glutamate. The biosynthesis of serine takes place in a three-step process and begins with 3-phosphoglycerate (an intermediate in glycolysis), and affords this amino acid after oxidation, transamination and hydrolysis steps. Cysteine and glycine are in each case produced from serine, namely the former by condensation of homocysteine with serine, and the latter by transfer of the side chain alpha-carbon atom to tetrahydrofolate, in a reaction catalyzed by serine transhydroxymethylase. Phenylalanine and tyrosine are synthesized from the precursors of the glycolysis and pentose phosphate pathway, erythrose 4-phosphate and phosphoenolpyruvate in a 9-step biosynthesis pathway, which only differs in the last two steps after the synthesis of prephenate. Tryptophan is likewise produced from these two starting molecules, but its synthesis takes place in an 11-step pathway. Tyrosine can also be prepared from phenylalanine in a reaction catalyzed by phenylalanine hydroxylase. Alanine, valine and leucine are in each case biosynthesis products of pyruvate, the end product of the glycolysis. Aspartate is formed from oxalacetate, an intermediate of the citrate cycle. Asparagine, methionine, threonine and lysine are in each case formed by conversion of aspartate. Isoleucine is formed from threonine. In a complex 9-step pathway, the formation of histidine takes place from 5-phosphoribosyl-1-pyrophosphate, an activated sugar.

Amino acids whose amount exceeds the protein biosynthesis need of the cell cannot be stored, and instead of this are broken down, so that intermediates for the main metabolic pathways of the cell are made available (for a general survey see Stryer, L., Biochemistry, 3rd ed. chap. 21 “Amino Acid Degradation and the Urea Cycle”; pp 495-516 (1988)). Although the cell is able to convert undesired amino acids to useful metabolic intermediates, the amino acid production with respect to the energy, the precursor molecules and the enzymes necessary for their synthesis is complicated. It is therefore not surprising that amino acid biosynthesis is regulated by feedback inhibition, the presence of a certain amino acid slowing or completely ending its own production (for a general survey of the feedback mechanism in amino acid biosynthesis pathways, see Stryer, L., Biochemistry, 3rd ed., chap. 24, “Biosynthesis of Amino Acids and Heme”, pp. 575-600 (1988)). The output of a certain amino acid is therefore restricted by the amount of this amino acid in the cell.

### II. Vitamins, Carotenoids, Cofactors and Nutraceuticals-Metabolism and Uses

Vitamins, carotenoids, cofactors and nutraceuticals comprise a further group of molecules. Higher animals have lost the ability to synthesize these and must thus assimilate them, although they are easily synthesized by other organisms, such as bacteria. These molecules are either biologically active molecules per se or precursors of biologically active substances, which serve as electron carriers or intermediates in a number of metabolic pathways. In addition to their nutritional value, these compounds also have a significant industrial value as colorants, antioxidants and catalysts or other processing aids. (For a general survey on the structure, activity and the industrial applications of these compounds see, for example, Ullmann's Encyclopedia of Industrial Chemistry, “Vitamins”, vol. A27, pp. 443-613, VCH: Weinheim, 1996). The term “vitamin” is known in the specialist field and comprises nutrients which are needed by an organism for normal function, but cannot be synthesized by this organism itself. The group consisting of the vitamins can comprise cofactors and nutraceutical compounds. The term “cofactor” comprises nonprotein-like compounds, which are necessary for the occurrence of normal enzyme activity. These compounds can be organic or inorganic; the cofactor molecules according to the invention are preferably organic. The term “nutraceutical” comprises food additives which are health-promoting in plants and animals, in particular humans. Examples of such molecules are vitamins, antioxidants and also certain lipids (e.g. polyunsaturated fatty acids).

Preferred fine chemicals or biosynthetic products which can be prepared in plants of the genus Tagetes, in particular in petals of the flowers of the plants of the genus Tagetes, are carotenoids, such as, for example, phytoene, lycopene, lutein, zeaxanthin, astaxanthin, canthaxanthin, echinenone, 3-hydroxyechinenone, 3′-hydroxyechinenone, adonirubin, violaxanthin and adonixanthin.

Particularly preferred carotenoids are ketocarotenoids, such as, for example, astaxanthin, canthaxanthin, echinenone, 3-hydroxyechinenone, 3′-hydroxyechinenone, adonirubin and adonixanthin.

The biosynthesis of these molecules in organisms which are capable of their production, such as bacteria, has been comprehensively characterized (Ullmann's Encyclopedia of Industrial Chemistry, “Vitamins”, vol. A27, pp. 443-613, VCH: Weinheim, 1996, Michal, G. (1999) Biochemical Pathways: An Atlas of Biochemistry and Molecular Biology, John Wiley & Sons; Ong, A. S., Niki, E. and Packer, L. (1995) “Nutrition, Lipids, Health and Disease” Proceedings of the UNESCO/Confederation of Scientific and Technological Associations in Malaysia and the Society for free Radical Research-Asia, held on Sep. 1-3, 1994 in Penang, Malaysia, AOCS Press, Champaign, Ill. X, 374 S).

Thiamine (vitamin B₁) is formed by chemical coupling of pyrimidine and thiazole units. Riboflavin (vitamin B₂) is synthesized from guanosine 5′-triphosphate (GTP) and ribose 5′-phosphate. Riboflavin is in turn employed for the synthesis of flavine mononucleotide (FMN) and flavine adenine dinucleotide (FAD). The family of compounds which together are described as “vitamin B6” (for example pyridoxine, pyridoxamine, pyridoxal 5′-phosphate and the commercially used pyridoxine hydrochloride), are all derivatives of the common structural unit 5-hydroxy-6-methylpyridine. Panthothenate (pantothenic acid, R-(+)-N-(2,4-dihydroxy-3,3-dimethyl-1-oxobutyl)-R-alanine) can be prepared either by chemical synthesis or by fermentation. The last steps in pantothenate biosynthesis consist of the ATP-driven condensation of R-alanine and pantoic acid. The enzymes responsible for the biosynthesis steps for the conversion to pantoic acid, to R-alanine and for the condensation to pantothenic acid are known. The metabolically active form of pantothenate is coenzyme A, whose biosynthesis proceeds via 5 enzymatic steps. Pantothenate, pyridoxal 5′-phosphate, cysteine and ATP are the precursors of coenzyme A. These enzymes catalyze not only the formation of pantothenate, but also the production of (R)-pantoic acid, (R)-pantolactone, (R)-panthenol (provitamin B₅), pantethein (and its derivatives) and coenzyme A.

The biosynthesis of biotin from the precursor molecule pimeloyl-CoA in microorganisms has been investigated in detail, and several of the genes involved have been identified. It has emerged that many of the corresponding proteins are involved in the Fe cluster synthesis and belong to the class of the nifS proteins. Lipoic acid is derived from octanoic acid and serves as a coenzyme in energy metabolism, where it is a constituent of the pyruvate dehydrogenase complex and of the alpha-ketoglutarate dehydrogenase complex. The folates are a group of substances which are all derived from folic acid, which in turn is derived from L-glutamic acid, p-amino-benzoic acid and 6-methylpterin. The biosynthesis of folic acid and its derivatives, starting from the metabolic intermediates guanosine 5′-triphosphate (GTP), L-glutamic acid and p-amino-benzoic acid, has been investigated in detail in certain microorganisms.

Corrinoids (such as the cobalamines and in particular vitamin B₁₂) and the porphyrins belong to a group of chemicals which are distinguished by a tetrapyrrole ring system. The biosynthesis of vitamin B₁₂ is sufficiently complex that it has still not been completely characterized, but in the meantime a majority of the enzymes and substrates involved is known. Nicotinic acid (nicotinate) and nicotinamide are pyridine derivatives which are also described as “niacin”. Niacin is the precursor of the important coenzyme NAD (nicotinamide adenine dinucleotide) and NADP (nicotinamide adenine dinucleotide phosphate) and their reduced forms.

The production of these compounds on the large-scale is based in the main on cell-free chemical syntheses, although some of these chemicals have also been produced by large-scale culture of microorganisms, such as riboflavin, vitamin B₆, pantothenate and biotin. Only vitamin B₁₂ is produced solely by fermentation on account of the complexity of its synthesis. In-vitro processes require a considerable expenditure of materials and time and frequently of high costs.

### III. Purine, Pyrimidine, Nucleoside and Nucleotide Metabolism and Uses

Genes for purine and pyrimidine metabolism and their corresponding proteins are important targets for the therapy of tumors and virus infections. The term “purine” or “pyrimidine” comprises nitrogen-containing bases, which are a constituent of the nucleic acids, coenzymes and nucleotides. The term “nucleotide” comprises the basic structural units of the nucleic acid molecules, which comprise a nitrogen-containing base, a pentose sugar (in RNA the sugar is ribose, in DNA the sugar is D-deoxyribose) and phosphoric acid. The term “nucleoside” comprises molecules which serve as precursors of nucleotides, but which in contrast to the nucleotides contain no phosphoric acid unit. By inhibition of the biosynthesis of these molecules or their mobilization for the formation of nucleic acid molecules it is possible to inhibit the RNA and DNA synthesis; if this activity is inhibited in cancerogenic cells in a targeted manner, the division and replication ability of tumor cells can be inhibited.

There are moreover nucleotides which form no nucleic acid molecules, but serve as energy stores (i.e. AMP) or as coenzymes (i.e. FAD and NAD).

Several publications have described the use of these chemicals for these medicinal indications, in which the purine and/or pyrimidine metabolism is influenced (for example Christopherson, R. I. and Lyons, S. D. (1990) “Potent inhibitors of de novo pyrimidine and purine biosynthesis as chemotherapeutic agents”, Med. Res. Reviews 10: 505-548). Investigations on enzymes which are involved in purine and pyrimidine metabolism have concentrated on the development of new medicaments, which can be used, for example, as immunosuppressive agents or antiproliferants (Smith, J. L. “Enzymes in Nucleotide Synthesis” Curr. Opin. Struct. Biol. 5 (1995) 752-757; Biochem. Soc. Transact. 23 (1995) 877-902). The purine and pyrimidine bases, nucleosides and nucleotides, however, also have other use possibilities: as intermediates in the biosynthesis of various fine chemicals (e.g. thiamine, S-adenosylmethionine, folates or riboflavin), as energy sources for the cell (for example ATP or GTP) and, for chemicals themselves, are usually used as flavor enhancers (for example IMP or GMP) or for many medicinal applications (see, for example, Kuninaka, A., (1996) “Nucleotides and Related Compounds in Biotechnology vol. 6, Rehm et al., ed. VCH: Weinheim, pp. 561-612). Enzymes which are involved in purine, pyrimidine, nucleoside or nucleotide metabolism are also used more and more highly as targets against which chemicals for plant protection, including fungicides, herbicides and insecticides, are developed.

The metabolism of these compounds in bacteria has been characterized (for general surveys see, for example, Zalkin, H. and Dixon, J. E. (1992) “De novo purine nucleotide biosynthesis” in Progress in Nucleic Acids Research and Molecular biology, vol. 42, Academic Press, pp. 259-287; and Michal, G. (1999) “Nucleotides and Nucleosides”; chap. 8 in: Biochemical Pathways: An Atlas of Biochemistry and Molecular Biology, Wiley, New York). Purine metabolism, the subject of intensive research, is essential for the normal functioning of the cell. Defective purine metabolism in higher animals can cause serious disorders, for example gout. The purine nucleotides are synthesized from ribose 5-phosphate via a series of steps by means of the intermediate compound inosine 5′-phosphate (IMP), which leads to the production of guanosine 5′-mono-phosphate (GMP) or adenosine 5′-monophosphate (AMP), from which the triphosphate forms used as nucleotides can readily be prepared. These compounds are also used as energy stores, so that their breakdown supplies energy for many different biochemical processes in the cell. Pyrimidine biosynthesis takes place via the formation of uridine 5′-monophosphate (UMP) from ribose 5-phosphate. UMP in turn is converted to cytidine 5′-triphosphate (CTP). The deoxy forms of all nucleotides are prepared in a single-step reduction reaction of the diphosphate ribose form of the nucleotide to the diphosphate deoxyribose form of the nucleotide. After phosphorylation, these molecules can participate in DNA synthesis.

### IV. Trehalose Metabolism and Uses

Trehalose consists of two glucose molecules, which are linked to one another. It is usually used in the foodstuffs industry as a sweetener, as an additive for dried or frozen foods and in drinks. It is, however, also used in the pharmaceutical industry, the cosmetics and biotechnology industry (see, for example, Nishimoto et al., (1998) U.S. Pat. No. 5,759,610; Singer, M. A. and Lindquist, S. Trends Biotech. 16 (1998) 460-467; Paiva, C. L. A. and Panek, A. D. Biotech Ann. Rev. 2 (1996) 293-314; and Shiosaka, M. J. Japan 172 (1997) 97-102). Trehalose is produced by enzymes of many microorganisms and released naturally into the surrounding medium, from which it can be recovered by processes known in the specialist field.

Particularly preferred biosynthetic products are selected from the group consisting of organic acids, proteins, nucleotides and nucleosides, both proteinogenic and nonproteinogenic amino acids, lipids and fatty acids, diols, carbohydrates, aromatic compounds, vitamins and cofactors, enzymes and proteins.

Preferred organic acids are tartaric acid, itaconic acid and diaminopimelic acid.

Preferred nucleosides and nucleotides are described, for example, in Kuninaka, A. (1996) Nucleotides and related compounds, pp. 561-612, in Biotechnology vol. 6, Rehm et al., ed. VCH: Weinheim and the citations contained therein.

Preferred biosynthetic products are furthermore lipids, saturated and unsaturated fatty acids, such as, for example, arachidonic acid, diols such as, for example, propanediol and butanediol, carbohydrates, such as, for example, hyaluronic acid and trehalose, aromatic compounds, such as, for example, aromatic amines, vanillin and indigo, vitamins and cofactors, such as are described, for example, in Ullmann's Encyclopedia of Industrial Chemistry, vol. A27, “Vitamins”, pp. 443-613 (1996) VCH: Weinheim and the citations comprised therein; and Ong, A. S., Niki, E. and Packer, L. (1995) “Nutrition, Lipids, Health and Disease” Proceedings of the UNESCO/Confederation of Scientific and Technological Associations in Malaysia and the Society for Free Radical Research—Asia, held on Sep. 1-3, 1994 in Penang, Malysia, AOCS Press (1995)), enzyme polyketides (Cane et al. (1998) Science 282: 63-68), and all other chemicals described by Gutcho (1983) in Chemicals by Fermentation, Noyes Data Corporation, ISBN: 0818805086 and the references stated therein.

Particularly preferred genes which are expressed with the promoters according to the invention in plants of the genus Tagetes are accordingly genes selected are from the group consisting of nucleic acids encoding a protein from the biosynthesis pathway of proteinogenic and nonproteinogenic amino acids, nucleic acids encoding a protein from the biosynthesis pathway of nucleotides and nucleosides, nucleic acids encoding a protein from the biosynthesis pathway of organic acids, nucleic acids encoding a protein from the biosynthesis pathway of lipids and fatty acids, nucleic acids encoding a protein from the biosynthesis pathway of diols, nucleic acids encoding a protein from the biosynthesis pathway of carbohydrates, nucleic acids encoding a protein from the biosynthesis pathway of aromatic compound, nucleic acids encoding a protein from the biosynthesis pathway of vitamins, nucleic acids encoding a protein from the biosynthesis pathway of carotenoids, in particular ketocarotenoids, nucleic acids encoding a protein from the biosynthesis pathway of cofactors and nucleic acids encoding a protein from the biosynthesis pathway of enzymes.

Preferred fine chemicals or biosynthetic products which can be produced in plants of the genus Tagetes, in particular in petals of the flowers of the plants of the genus Tagetes, are carotenoids, such as, for example, phytoene, lycopene, lutein, zeaxanthin, astaxanthin, canthaxanthin, echinenone, 3-hydroxyechinenone, 3′-hydroxyechinenone, adonirubin, violaxanthin and adonixanthin. Particularly preferred carotenoids are ketocarotenoids, such as, for example, astaxanthin, canthaxanthin, echinenone, 3-hydroxyechinenone, 3′-hydroxyechinenone, adonirubin and adonixanthin.

Very particularly preferred genes which are expressed using the promoters according to the invention in plants of the genus Tagetes are accordingly genes which encode proteins from the biosynthesis pathway of carotenoids.

Genes selected from the group consisting of nucleic acids encoding a beta-hydroxylase, nucleic acids encoding a beta-cyclase, nucleic acids encoding an epsilon-cyclase, nucleic acids encoding a zeaxanthin-epoxidase, nucleic acids encoding a antheraxanthin-epoxidase, nucleic acids encoding a neoxanthin-synthase, nucleic acids encoding an HMG-CoA reductase, nucleic acids encoding an (E)-4-hydroxy-3-methylbut-2-enyl-diphosphate reductase, nucleic acids encoding a 1-deoxy-D-xylose-5-phosphate synthase, nucleic acids encoding a 1-deoxy-D-xylose-5-phosphate reductoisomerase, nucleic acids encoding an isopentenyl diphosphate delta-isomerase, nucleic acids encoding a geranyl diphosphate synthase, nucleic acids encoding a farnesyl diphosphate synthase, nucleic acids encoding a geranylgeranyl diphosphate synthase, nucleic acids encoding a phytoene synthase, nucleic acids encoding a phytoene desaturase, (phytoenedehydrogenase), nucleic acids encoding a prephytoene synthase, nucleic acids encoding a zeta-carotene desaturase, nucleic acids encoding a crtlSO protein, nucleic acids encoding a 4-diphosphocytidyl-2-C-methyl-D-erythritol synthase, nucleic acids encoding a 4-diphosphocytidyl-2-C-methyl-D-erythritol kinase, nucleic acids encoding a 2-Methyl-D-erythritol cyclophosphate-synthase, nucleic acids encoding a Hydroxymethylbutenyldiphosphate-synthase, nucleic acids encoding an FtsZ protein and nucleic acids encoding a MinD protein are particularly preferred.

A beta-cyclase is understood as meaning a protein which has the enzymatic activity to convert a terminal, linear residue of lycopene to a beta-ionone ring.

In particular, a beta-cyclase is understood as meaning a protein which has the enzymatic activity to convert gamma-carotene to beta-carotene.

Examples of beta-cyclase genes are nucleic acids encoding a beta-cyclase from tomato (Accession X86452), and beta-cyclases of the following accession numbers:

| | |
|---|---|
| S66350 | lycopene beta-cyclase (EC 5.5.1.-) - tomato |
| CAA60119 | lycopene synthase [Capsicum annuum] |
| S66349 | lycopene beta-cyclase (EC 5.5.1.-) - common tobacco |
| CAA57386 | lycopene cyclase [Nicotiana tabacum] |
| AAM21152 | lycopene beta-cyclase [Citrus sinensis] |
| AAD38049 | lycopene cyclase [Citrus × paradisi] |
| AAN86060 | lycopene cyclase [Citrus unshiu] |
| AAF44700 | lycopene beta-cyclase [Citrus sinensis] |
| AAK07430 | lycopene beta-cyclase [Adonis palaestina] |
| AAG10429 | beta cyclase [Tagetes erecta] |
| AAA81880 | lycopene cyclase |
| AAB53337 | Lycopene beta cyclase |
| AAL92175 | beta-lycopene cyclase [Sandersonia aurantiaca] |
| CAA67331 | lycopene cyclase [Narcissus pseudonarcissus] |
| AAM45381 | beta cyclase [Tagetes erecta] |
| AAO18661 | lycopene beta-cyclase [Zea mays] |
| AAG21133 | chromoplast-specific lycopene beta-cyclase |
| | [Lycopersicon esculentum] |
| AAF18989 | lycopene beta-cyclase [Daucus carota] |
| ZP_001140 | hypothetical protein [Prochlorococcus marinus str. |
| | MIT9313] |
| ZP_001050 | hypothetical protein [Prochlorococcus marinus subsp. |
| | pastoris str. CCMP1378] |
| ZP_001046 | hypothetical protein [Prochlorococcus marinus subsp. |
| | pastoris str. CCMP1378] |
| ZP_001134 | hypothetical protein [Prochlorococcus marinus str. |
| | MIT9313] |
| ZP_001150 | hypothetical protein [Synechococcus sp. WH 8102] |
| AAF10377 | lycopene cyclase [Deinococcus radiodurans] |
| BAA29250 | 393aa long hypothetical protein [Pyrococcus horikoshii] |
| BAC77673 | lycopene beta-monocyclase [marine bacterium P99-3] |
| AAL01999 | lycopene cyclase [Xanthobacter sp. Py2] |
| ZP_000190 | hypothetical protein [Chloroflexus aurantiacus] |
| ZP_000941 | hypothetical protein [Novosphingobium aromaticivorans] |
| AAF78200 | lycopene cyclase [Bradyrhizobium sp. ORS278] |
| BAB79602 | crtY [Pantoea agglomerans pv. milletiae] |
| CAA64855 | lycopene cyclase [Streptomyces griseus] |
| AAA21262 | dycopene cyclase [Pantoea agglomerans] |
| C37802 | crtY protein - Erwinia uredovora |
| BAB79602 | crtY [Pantoea agglomerans pv. milletiae] |
| AAA64980 | lycopene cyclase [Pantoea agglomerans] |
| AAC44851 | lycopene cyclase |
| BAA09593 | Lycopene cyclase [Paracoccus sp. MBIC1143] |
| ZP_000941 | hypothetical protein [Novosphingobium aromaticivorans] |
| CAB56061 | lycopene beta-cyclase [Paracoccus marcusii] |
| BAA20275 | lycopene cyclase [Erythrobacter longus] |
| ZP_000570 | hypothetical protein [Thermobifida fusca] |
| ZP_000190 | hypothetical protein [Chloroflexus aurantiacus] |
| AAK07430 | lycopene beta-cyclase [Adonis palaestina] |
| CAA67331 | lycopene cyclase [Narcissus pseudonarcissus] |
| AAB53337 | Lycopene beta cyclase |
| BAC77673 | lycopene beta-monocyclase [marine bacterium P99-3] |

A particularly preferred beta-cyclase is furthermore the chromoplast-specific beta-cyclase from tomato (AAG21133)

A hydroxylase is understood as meaning a protein which has the enzymatic activity to introduce a hydroxyl group on the optionally substituted, R-ionone ring of carotenoids.

In particular, a hydroxylase is understood as meaning a protein which has the enzymatic activity to convert-carotene to zeaxanthin or canthaxanthin to astaxanthin.

Examples of a hydroxylase gene are:

a nucleic acid encoding a hydroxylase from Haematococcus pluvialis, Accession AX038729, WO 0061764); (nucleic acid: SEQ ID NO: 49, protein: SEQ ID NO: 50),
and hydroxylases of the following accession numbers:
|emb|CAB55626.1, CAA70427.1, CAA70888.1, CAB55625.1, AF499108_{—}1, AF315289_{—}1, AF296158_{—}1, AAC49443.1, NP_{—}194300.1, NP_{—}200070.1, AAG10430.1, CAC06712.1, AAM88619.1, CAC95130.1, AAL80006.1, AF162276_{—}1, AAO53295.1, AAN85601.1, CRTZ_ERWHE, CRTZ_PANAN, BAB79605.1, CRTZ_ALCSP, CRTZ_AGRAU, CAB56060.1, ZP_{—}00094836.1, AAC44852.1, BAC77670.1, NP_{—}745389.1, NP_{—}344225.1, NP_{—}849490.1, ZP_{—}00087019.1, NP_{—}503072.1, NP_{—}852012.1, NP_{—}115929.1, ZP_{—}00013255.1

A particularly preferred hydroxylase is furthermore the hydroxylase from tomato (Accession Y14810, CrtR-b2) (nucleic acid: SEQ ID NO: 51; protein: SEQ ID NO. 52).

An HMG-CoA reductase is understood as meaning a protein which has the enzymatic activity to convert 3-hydroxy-3-methyl-glutarylcoenzyme A to mevalonate.

An (E)-4-hydroxy-3-methylbut-2-enyl-diphosphate reductase is understood as meaning a protein which has the enzymatic activity to convert (E)-4-hydroxy-3-methylbut-2-enyl diphosphate to isopentenyl diphosphate and dimethylallyl diphosphates.

A 1-deoxy-D-xylose-5-phosphate synthase is understood as meaning a protein which has the enzymatic activity to convert hydroxyethyl-ThPP and glyceraldehyde 3-phosphate to 1-deoxy-D-xylose 5-phosphate.

A 1-deoxy-D-xylose-5 phosphate reductoisomerase is understood as meaning a protein which has the enzymatic activity to convert 1-deoxy-D-xylose 5-phosphate to 2-C-methyl-D-erythritol 4-phosphate.

An isopentenyl diphosphate delta-isomerase is understood as meaning a protein which has the enzymatic activity to convert isopentenyl diphosphate to dimethylallyl phosphate.

A geranyl diphosphate synthase is understood as meaning a protein which has the enzymatic activity to convert isopentenyl diphosphate and dimethylallyl phosphate to geranyl diphosphate.

A farnesyl diphosphate synthase is understood as meaning a protein which has the enzymatic activity to convert sequentially 2 molecules of isopentenyl diphosphate using dimethylallyl diphosphate and the resulting geranyl diphosphate to farnesyl diphosphate.

A geranylgeranyl diphosphate synthase is understood as meaning a protein which has the enzymatic activity to convert farnesyl diphosphate and isopentenyl diphosphate to geranylgeranyl diphosphate.

A phytoene synthase is understood as meaning a protein which has the enzymatic activity to convert geranylgeranyl diphosphate to phytoene.

A phytoene desaturase is understood as meaning a protein which has the enzymatic activity to convert phytoene to phytofluene and/or phytofluene to zeta-carotene.

A zeta-carotene desaturase is understood as meaning a protein which has the enzymatic activity to convert zeta-carotene to neurosporin and/or neurosporin to lycopene.

A crtlSO protein is understood as meaning a protein which has the enzymatic activity to convert 7,9,7′,9′-tetra-cis-lycopene to all-trans-lycopene.

An FtsZ protein is understood as meaning a protein which has a cell division and plastidic division-promoting action and has homologies to tubulin proteins.

A MinD protein is understood as meaning a protein which has a multifunctional role in cell division. It is a membrane-associated ATPase and can show within the cell an oscillating motion from pole to pole.

Examples of HMG-CoA reductase genes are:

a nucleic acid encoding an HMG-CoA reductase from Arabidopsis thaliana, Accession NM_{—}106299;
and further HMG-CoA reductase genes from other organisms having the following accession numbers:

P54961, P54870, P54868, P54869, O02734, P22791, P54873, P54871, P23228, P13704, P54872, Q01581, P17425, P54874, P54839, P14891, P34135, O64966, P29057, P48019, P48020, P12683, P43256, Q9XEL8, P34136, O64967, P29058, P48022, Q41437, P12684, Q00583, Q9XHL5, Q41438, Q9YAS4, O76819, O28538, Q9Y7D2, P54960, O51628, P48021, Q03163, P00347, P14773, Q12577, Q59468, P04035, O24594, P09610, Q58116, O26662, Q01237, Q01559, Q12649, O74164, O59469, P51639, Q10283, O08424, P20715, P13703, P13702, Q96UG4, Q8SQZ9, O15888, Q9TUM4, P93514, Q39628, P93081, P93080, Q944T9, Q40148, Q84mM0, Q84LS3, Q9Z9N4, Q9KLM0

Examples of (E)-4-hydroxy-3-methylbut-2-enyl diphosphate reductase genes are:

A nucleic acid encoding an (E)-4-hydroxy-3-methylbut-2-enyl diphosphate reductase from Arabidopsis thaliana (IytB/ISPH), ACCESSION AY168881, and further (E)-4-hydroxy-3-methylbut-2-enyl-diphosphate reductase genes from other organisms having the following accession numbers:

T04781, AF270978_{—}1, NP_{—}485028.1, NP_{—}442089.1, NP_{—}681832.1, ZP_{—}00110421.1, ZP_{—}00071594.1, ZP_{—}00114706.1, ISPH_SYNY3, ZP_{—}00114087.1, ZP_{—}00104269.1, AF398145_{—}1, AF398146_{—}1, AAD55762.1, AF514843_{—}1, NP_{—}622970.1, NP_{—}348471.1, NP_{—}562001.1, NP_{—}223698.1, NP_{—}781941.1, ZP_{—}00080042.1, NP_{—}859669.1, NP_{—}214191.1, ZP_{—}00086191.1, ISPH_VIBCH, NP_{—}230334.1, NP_{—}742768.1, NP_{—}302306.1, ISPH_MYCLE, NP_{—}602581.1, ZP_{—}00026966.1, NP_{—}520563.1, NP_{—}253247.1, NP_{—}282047.1, ZP_{—}00038210.1, ZP_{—}00064913.1, CAA61555.1, ZP_{—}00125365.1, ISPH_ACICA, EAA24703.1, ZP_{—}00013067.1, ZP_{—}00029164.1, NP_{—}790656.1, NP_{—}217899.1, NP_{—}641592.1, NP_{—}636532.1, NP_{—}719076.1, NP_{—}660497.1, NP_{—}422155.1, NP_{—}715446.1, ZP_{—}00090692.1, NP_{—}759496.1, ISPH_BURPS, ZP_{—}00129657.1, NP_{—}215626.1, NP_{—}335584.1, ZP_{—}00135016.1, NP_{—}789585.1, NP_{—}787770.1, NP_{—}769647.1, ZP_{—}00043336.1, NP_{—}242248.1, ZP_{—}00008555.1, NP_{—}246603.1, ZP_{—}00030951.1, NP_{—}670994.1, NP_{—}404120.1, NP_{—}540376.1, NP_{—}733653.1, NP_{—}697503.1, NP_{—}840730.1, NP_{—}274828.1, NP_{—}796916.1, ZP_{—}00123390.1, NP_{—}824386.1, NP_{—}737689.1, ZP_{—}00021222.1, NP_{—}757521.1, NP_{—}390395.1, ZP_{—}00133322.1, CAD76178.1, NP_{—}600249.1, NP_{—}454660.1, NP_{—}712601.1, NP_{—}385018.1, NP_{—}751989.1

Examples of 1-deoxy-D-xylose-5-phosphate synthase genes are:

A nucleic acid encoding a 1-deoxy-D-xylose-5-phosphate synthase from Lycopersicon esculentum, ACCESSION #AF143812 and further 1-deoxy-D-xylose-5-phosphate synthase genes from other organisms having the following accession numbers: AF143812_{—}1, DXS_CAPAN, CAD22530.1, AF182286_{—}1, NP_{—}193291.1, T52289, AAC49368.1, AAP14353.1, D71420, DXS_ORYSA, AF443590_{—}1, BAB02345.1, CAA09804.2, NP_{—}850620.1, CAD22155.2, AAM65798.1, NP_{—}566686.1, CAD22531.1, AAC33513.1, CAC08458.1, AAG10432.1, T08140, AAP14354.1, AF428463_{—}1, ZP_{—}00010537.1, NP_{—}769291.1, AAK59424.1, NP_{—}107784.1, NP_{—}697464.1, NP_{—}540415.1, NP_{—}196699.1, NP_{—}384986.1, ZP_{—}00096461.1, ZP_{—}00013656.1, NP_{—}353769.1, BAA83576.1, ZP_{—}00005919.1, ZP_{—}00006273.1, NP_{—}420871.1, AAM48660.1, DXS_RHOCA, ZP_{—}00045608.1, ZP_{—}00031686.1, NP_{—}841218.1, ZP_{—}00022174.1, ZP_{—}00086851.1, NP_{—}742690.1, NP_{—}520342.1, ZP_{—}00082120.1, NP_{—}790545.1, ZP_{—}00125266.1, CAC17468.1, NP_{—}252733.1, ZP_{—}00092466.1, NP_{—}439591.1, NP_{—}414954.1, NP_{—}752465.1, NP_{—}622918.1, NP_{—}286162.1, NP_{—}836085.1, NP_{—}706308.1, ZP_{—}00081148.1, NP_{—}797065.1, NP_{—}213598.1, NP_{—}245469.1, ZP_{—}00075029.1, NP_{—}455016.1, NP_{—}230536.1, NP_{—}459417.1, NP_{—}274863.1, NP_{—}283402.1, NP_{—}759318.1, NP_{—}406652.1, DXS_SYNLE, DXS_SYNP7, NP_{—}440409.1, ZP_{—}00067331.1, ZP_{—}00122853.1, NP_{—}717142.1, ZP_{—}00104889.1, NP_{—}243645.1, NP_{—}681412.1, DXS_SYNEL, NP_{—}637787.1, DXS_CHLTE, ZP_{—}00129863.1, NP_{—}661241.1, DXS_XANCP, NP_{—}470738.1, NP_{—}484643.1, ZP_{—}00108360.1, NP_{—}833890.1, NP_{—}846629.1, NP_{—}658213.1, NP_{—}642879.1, ZP_{—}00039479.1, ZP_{—}00060584.1, ZP_{—}00041364.1, ZP_{—}00117779.1, NP_{—}299528.1

Examples of 1-deoxy-D-xylose-5-phosphate reductoisomerase genes are:

A nucleic acid encoding a 1-deoxy-D-xylose-5-phosphate reductoisomerase from Arabidopsis thaliana, ACCESSION #AF148852, and further 1-deoxy-D-xylose-5-phosphate reductoisomerase genes from other organisms having the following accession numbers:

AF148852, AY084775, AY054682, AY050802, AY045634, AY081453, AY091405, AY098952, AJ242588, AB009053, AY202991, NP_{—}201085.1, T52570, AF331705_{—}1, BAB16915.1, AF367205_{—}1, AF250235_{—}1, CAC03581.1, CAD22156.1, AF182287_{—}1, DXR_MENP₁, ZP_{—}00071219.1, NP_{—}488391.1, ZP_{—}00111307.1, DXR_SYNLE, AAP56260.1, NP_{—}681831.1, NP_{—}442113.1, ZP_{—}00115071.1, ZP_{—}00105106.1, ZP_{—}00113484.1, NP_{—}833540.1, NP_{—}657789.1, NP_{—}661031.1, DXR_BACHD, NP_{—}833080.1, NP_{—}845693.1, NP_{—}562610.1, NP_{—}623020.1, NP_{—}810915.1, NP_{—}243287.1, ZP_{—}00118743.1, NP_{—}464842.1, NP_{—}470690.1, ZP_{—}00082201.1, NP_{—}781898.1, ZP_{—}00123667.1, NP_{—}348420.1, NP_{—}604221.1, ZP_{—}00053349.1, ZP_{—}00064941.1, NP_{—}246927.1, NP_{—}389537.1, ZP_{—}00102576.1, NP_{—}519531.1, AF124757_{—}19, DXR_ZYMMO, NP_{—}713472.1, NP_{—}459225.1, NP_{—}454827.1, ZP_{—}00045738.1, NP_{—}743754.1, DXR_PSEPK, ZP_{—}00130352.1, NP_{—}702530.1, NP_{—}841744.1, NP_{—}438967.1, AF514841_{—}1, NP_{—}706118.1, ZP_{—}00125845.1, NP_{—}404661.1, NP_{—}285867.1, NP_{—}240064.1, NP_{—}414715.1, ZP_{—}00094058.1, NP_{—}791365.1, ZP_{—}00012448.1, ZP_{—}00015132.1, ZP_{—}00091545.1, NP_{—}629822.1, NP_{—}771495.1, NP_{—}798691.1, NP_{—}231885.1, NP_{—}252340.1, ZP_{—}00022353.1, NP_{—}355549.1, NP_{—}420724.1, ZP_{—}00085169.1, EAA17616.1, NP_{—}273242.1, NP_{—}219574.1, NP_{—}387094.1, NP_{—}296721.1, ZP_{—}00004209.1, NP_{—}823739.1, NP_{—}282934.1, BAA77848.1, NP_{—}660577.1, NP_{—}760741.1, NP_{—}641750.1, NP_{—}636741.1, NP_{—}829309.1, NP_{—}298338.1, NP_{—}444964.1, NP_{—}717246.1, NP_{—}224545.1, ZP_{—}00038451.1, DXR_KITGR, NP_{—}778563.1.

Examples of isopentenyl diphosphate delta-isomerase genes are:

A nucleic acid encoding an isopentenyl diphosphate delta-isomerase from Adonis palaestina clone AplPI28, (ipiAa1), ACCESSION #AF188060, published by Cunningham, F. X. Jr. and Gantt, E.: Identification of multi-gene families encoding isopentenyl diphosphate isomerase in plants by heterologous complementation in Escherichia coli, Plant Cell Physiol. 41 (1), 119-123 (2000) and further isopentenyl diphosphate-isomerase genes from other organisms having the following accession numbers:

Q38929, O48964, Q39472, Q13907, O35586, P58044, O42641, O35760, Q10132, P15496, Q9YB30, Q8YNH4, Q42553, O27997, P50740, O51627, O48965, Q8KFR5, Q39471, Q39664, Q9RVE2, Q01335, Q9HHE4, Q9BXS1, Q9 KWF6, Q9CIF5, Q88WB6, Q92BX2, Q8Y7A5, Q8TT35 Q9KK75, Q8NN99, Q8XD58, Q8FE75, Q46822, Q9HP40, P72002, P26173, Q9Z5D3, Q8Z3X9, Q8ZM82, Q9X7Q6, O13504, Q9HFW8, Q8NJL9, Q9UUQ1, Q9NH02, Q9M6K9, Q9M6K5, Q9FXR6, O81691, Q9S7C4, Q8S3L8, Q9M592, Q9M6K3, Q9M6K7, Q9FV48, Q9LLB6, Q9AVJ1, Q9AVG8, Q9M6K6, Q9AVJ5, Q9M6K2, Q9AYS5, Q9M6K8, Q9AVG7, Q8S3L7, Q8W250, Q94IE1, Q9AVI8, Q9AYS6, Q9SAY0, Q9M6K4, Q8GVZ0, Q84RZ8, Q8KZ12, Q8KZ66, Q8FND7, Q88QC9, Q8BFZ6, BAC26382, CAD94476.

Examples of geranyl diphosphate synthase genes are:

A nucleic acid encoding a geranyl diphosphate synthase from Arabidopsis thaliana, ACCESSION #Y17376, Bouvier, F., Suire, C., d'Harlingue, A., Backhaus, R. A. and Camara, B.; Molecular cloning of geranyl diphosphate synthase and compartmentation of monoterpene synthesis in plant cells, Plant J. 24 (2), 241-252 (2000) and further geranyl diphosphate synthase genes from other organisms having the following accession numbers:

Q9FT89, Q8LKJ2, Q9FSW8, Q8LKJ3, Q9SBR3, Q9SBR4, Q9FET8, Q8LKJ1, Q84LG1, Q9JK86

Examples of farnesyl diphosphate synthase genes are:

A nucleic acid encoding a farnesyl diphosphate synthase from Arabidopsis thaliana (FPS1), ACCESSION #U80605, published by Cunillera, N., Arro, M., Delourme, D., Karst, F., Boronat, A. and Ferrer, A.: Arabidopsis thaliana contains two differentially expressed farnesyl-diphosphate synthase genes, J. Biol. Chem. 271 (13), 7774-7780 (1996), and further farnesyl diphosphate synthase genes from other organisms having the following accession numbers

P53799, P37268, Q02769, Q09152, P49351, O24241, Q43315, P49352, O24242, P49350, P08836, P14324, P49349, P08524, O66952, Q08291, P54383, Q45220, P57537, Q8K9A0, P22939, P45204, O66126, P55539, Q9SWH9, Q9AVI7, Q9FRX2, Q9AYS7, Q941E8, Q9FXR9, Q9ZWF6, Q9FXR8, Q9AR37, O50009, Q941E9, Q8RVK7, Q8RVQ7, O04882, Q93RA8, Q93RB0, Q93RB4, Q93RB5, Q93RB3, Q93RB1, Q93RB2, Q920E5.

Examples of geranylgeranyl diphosphate synthase genes are:

A nucleic acid encoding a geranylgeranyl diphosphate synthase from Sinaps alba, ACCESSION #X98795, published by Bonk, M., Hoffmann, B., Von Lintig, J., Schledz, M., Al-Babili, S., Hobeika, E., Kleinig, H. and Beyer, P.: Chloroplast import of four carotenoid biosynthetic enzymes in vitro reveals differential fates prior to membrane binding and oligomeric assembly, Eur. J. Biochem. 247 (3), 942-950 (1997), and further geranylgeranyl diphosphate synthase genes from other organisms having the following accession numbers:

P22873, P34802, P56966, P80042, Q42698, Q92236, O95749, Q9WTN0, Q50727, P24322, P39464, Q9FXR3, Q9AYN2, Q9FXR2, Q9AVG6, Q9FRW4, Q9SXZ5, Q9AVJ7, Q9AYN1, Q9AVJ4, Q9FXR7, Q8LSC5, Q9AVJ6, Q8LSC4, Q9AVJ3, Q9SSU0, Q9SXZ6, Q9SST9, Q9AVJ0, Q9AVI9, Q9FRW3, Q9FXR5, Q94IF0, Q9FRX1, Q9K567, Q93RA9, Q93QX8, CAD95619, EAA31459

Examples of phytoene synthase genes are:

A nucleic acid encoding a phytoene synthase from Erwinia uredovora, ACCESSION # D90087; published by Misawa, N., Nakagawa, M., Kobayashi, K., Yamano, S., Izawa, Y., Nakamura, K. and Harashima, K.: Elucidation of the Erwinia uredovora carotenoid biosynthetic pathway by functional analysis of gene products expressed in Escherichia coli; J. Bacteriol. 172 (12), 6704-6712 (1990), and further phytoene synthase genes from other organisms having the following accession numbers:

CAB39693, BAC69364, AAF10440, CAA45350, BAA20384, AAM72615, BAC09112, CAA48922, P_{—}001091, CAB84588, AAF41518, CAA48155, AAD38051, AAF33237, AAG10427, AAA34187, BAB73532, CAC19567, AAM62787, CAA55391, AAB65697, AAM45379, CAC27383, AAA32836, AAK07735, BAA84763, P_{—}000205, AAB60314, P_{—}001163, P_{—}000718, AAB71428, AAA34153, AAK07734, CAA42969, CAD76176, CAA68575, P_{—}000130, P_{—}001142, CAA47625, CAA85775, BAC14416, CAA79957, BAC76563, P_{—}000242, P_{—}000551, AAL02001, AAK15621, CAB94795, AAA91951, P_{—}000448

Examples of phytoene desaturase genes are:

A nucleic acid encoding a phytoene desaturase from Erwinia uredovora, ACCESSION # D90087; published by Misawa, N., Nakagawa, M., Kobayashi, K., Yamano, S., Izawa, Y., Nakamura, K. and Harashima, K.: Elucidation of the Erwinia uredovora carotenoid biosynthetic pathway by functional analysis of gene products expressed in Escherichia coli; J. Bacteriol. 172 (12), 6704-6712 (1990), (nucleic acid: SEQ ID NO: 71, protein: SEQ ID NO: 72),

and further phytoene desaturase genes from other organisms having the following accession numbers:

AAL15300, A39597, CAA42573, AAK51545, BAB08179, CAA48195, BAB82461, AAK92625, CAA55392, AAG10426, AAD02489, AA024235, AAC12846, AAA99519, AAL38046, CAA60479, CAA75094, ZP_{—}001041, ZP_{—}001163, CAA39004, CAA44452, ZP_{—}001142, ZP_{—}000718, BAB82462, AAM45380, CAB56040, ZP_{—}001091, BAC09113, AAP79175, AAL80005, AAM72642, AAM72043, ZP_{—}000745, ZP_{—}001141, BAC07889, CAD55814, ZP_{—}001041, CAD27442, CAE00192, ZP_{—}001163, ZP_{—}000197, BAA18400, AAG10425, ZP_{—}001119, AAF13698, 2121278A, AAB35386, AAD02462, BAB68552, CAC85667, AAK51557, CAA12062, AAG51402, AAM63349, AAF85796, BAB74081, AAA91161, CAB56041, AAC48983, AAG14399, CAB65434, BAB73487, ZP_{—}001117, ZP_{—}000448, CAB39695, CAD76175, BAC69363, BAA17934, ZP_{—}000171, AAF65586, ZP_{—}000748, BAC07074, ZP_{—}001133, CAA64853, BAB74484, ZP_{—}001156, AAF23289, AAG28703, AAP09348, AAM71569, BAB69140, ZP_{—}000130, AAF41516, AAG18866, CAD95940, NP_{—}656310, AAG10645, ZP_{—}000276, ZP_{—}000192, ZP_{—}000186, AAM94364, EAA31371, ZP_{—}000612, BAC75676, AAF65582

Examples of zeta-carotene desaturase genes are:

A nucleic acid encoding a zeta-carotene desaturase from Narcissus pseudonarcissus, ACCESSION #AJ224683, published by Al-Babili, S., Oelschlegel, J. and Beyer, P.: A cDNA encoding for beta carotene desaturase (Accession No.AJ224683) from Narcissus pseudonarcissus L. (PGR98-103), Plant Physiol. 117, 719-719 (1998), and further zeta-carotene desaturase genes from other organisms having the following accession numbers:

Q9R6×4, Q38893, Q9SMJ3, Q9SE20, Q9ZTP4, O49901, P74306, Q9FV46, Q9RCT2, ZDS_NARPS, BAB68552.1, CAC85667.1, AF372617_{—}1, ZDS_TARER, CAD55814.1, CAD27442.1, 2121278A, ZDS_CAPAN, ZDS_LYCES, NP_{—}187138.1, AAM63349.1, ZDS_ARATH, AAA91161.1, ZDS_MAIZE, AAG14399.1, NP_{—}441720.1, NP_{—}486422.1, ZP_{—}00111920.1, CAB56041.1, ZP_{—}00074512.1, ZP_{—}00116357.1, NP_{—}681127.1, ZP_{—}00114185.1, ZP_{—}00104126.1, CAB65434.1, NP_{—}662300.1

Examples of crtlSO genes are:

A nucleic acid encoding a crtlSO from Lycopersicon esculentum; ACCESSION #AF416727, published by Isaacson, T., Ronen, G., Zamir, D. and Hirschberg, J.: Cloning of tangerine from tomato reveals a carotenoid isomerase essential for the production of beta-carotene and xanthophylls in plants; Plant Cell 14 (2), 333-342 (2002), and further crtlSO genes from other organisms having the following accession numbers:

### AAM53952

Examples of FtsZ genes are:

A nucleic acid encoding an FtsZ from Tagetes erecta, ACCESSION #AF251346, published by Moehs, C. P., Tian, L., Osteryoung, K. W. and Dellapenna, D.: Analysis of carotenoid biosynthetic gene expression during marigold petal development Plant Mol. Biol. 45 (3), 281-293 (2001), and further FtsZ genes from other organisms having the following accession numbers:

CAB89286.1, AF205858_{—}1, NP_{—}200339.1, CAB89287.1, CAB41987.1, AAA82068.1, T06774, AF383876_{—}1, BAC57986.1, CAD22047.1, BAB91150.1, ZP_{—}00072546.1, NP_{—}440816.1, T51092, NP_{—}683172.1, BAA85116.1, NP_{—}487898.1, JC4289, BAA82871.1, NP_{—}781763.1, BAC57987.1, ZP_{—}00111461.1, T51088, NP_{—}190843.1, ZP_{—}00060035.1, NP_{—}846285.1, AAL07180.1, NP_{—}243424.1, NP_{—}833626.1, AAN04561.1, AAN04557.1, CAD22048.1, T51089, NP_{—}692394.1, NP_{—}623237.1, NP_{—}565839.1, T51090, CAA07676.1, NP_{—}113397.1, T51087, CAC44257.1, E84778, ZP_{—}00105267.1, BAA82091.1, ZP_{—}00112790.1, BAA96782.1, NP_{—}348319.1, NP_{—}471472.1, ZP_{—}00115870.1, NP_{—}465556.1, NP_{—}389412.1, BAA82090.1, NP_{—}562681.1, AAM22891.1, NP_{—}371710.1, NP_{—}764416.1, CAB95028.1, FTSZ_STRGR, AF120117_{—}1, NP_{—}827300.1, JE0282, NP_{—}626341.1, AAC45639.1, NP_{—}785689.1, NP_{—}336679.1, NP_{—}738660.1, ZP_{—}00057764.1, AAC32265.1, NP_{—}814733.1, FTSZ_MYCKA, NP_{—}216666.1, CAA75616.1, NP_{—}301700.1, NP_{—}601357.1, ZP_{—}00046269.1, CAA70158.1, ZP_{—}00037834.1, NP_{—}268026.1, FTSZ_ENTHR, NP_{—}787643.1, NP_{—}346105.1, AAC32264.1, JC5548, AAC95440.1, NP_{—}710793.1, NP_{—}687509.1, NP_{—}269594.1, AAC32266.1, NP_{—}720988.1, NP_{—}657875.1, ZP_{—}00094865.1, ZP_{—}00080499.1, ZP_{—}00043589.1, JC7087, NP_{—}660559.1, AAC46069.1, AF179611_{—}14, AAC44223.1, NP_{—}404201.1.

Examples of MinD genes are:

A nucleic acid encoding a MinD from Tagetes erecta, ACCESSION #AF251019, published by Moehs, C. P., Tian, L., Osteryoung, K. W. and Dellapenna, D.: Analysis of carotenoid biosynthetic gene expression during marigold petal development; Plant Mol. Biol. 45 (3), 281-293 (2001), (nucleic acid: SEQ ID NO: 79, protein: SEQ ID NO: 80),

and further MinD genes having the following accession numbers:

NP_{—}197790.1, BAA90628.1, NP_{—}038435.1, NP_{—}045875.1, AAN33031.1, NP_{—}050910.1, CAB53105.1, NP_{—}050687.1, NP_{—}682807.1, NP_{—}487496.1, ZP_{—}00111708.1, ZP_{—}00071109.1, NP_{—}442592.1, NP_{—}603083.1, NP_{—}782631.1, ZP_{—}00097367.1, ZP_{—}00104319.1, NP_{—}294476.1, NP_{—}622555.1, NP_{—}563054.1, NP_{—}347881.1, ZP_{—}00113908.1, NP_{—}834154.1, NP_{—}658480.1, ZP_{—}00059858.1, NP_{—}470915.1, NP_{—}243893.1, NP_{—}465069.1, ZP_{—}00116155.1, NP_{—}390677.1, NP_{—}692970.1, NP_{—}298610.1, NP_{—}207129.1, ZP_{—}00038874.1, NP_{—}778791.1, NP_{—}223033.1, NP_{—}641561.1, NP_{—}636499.1, ZP_{—}00088714.1, NP_{—}213595.1, NP_{—}743889.1, NP_{—}231594.1, ZP_{—}00085067.1, NP_{—}797252.1, ZP_{—}00136593.1, NP_{—}251934.1, NP_{—}405629.1, NP_{—}759144.1, ZP_{—}00102939.1, NP_{—}793645.1, NP_{—}699517.1, NP_{—}460771.1, NP_{—}860754.1, NP_{—}456322.1, NP_{—}718163.1, NP_{—}229666.1, NP_{—}357356.1, NP_{—}541904.1, NP_{—}287414.1, NP_{—}660660.1, ZP_{—}00128273.1, NP_{—}103411.1, NP_{—}785789.1, NP_{—}715361.1, AF149810_{—}1, NP_{—}841854.1, NP_{—}437893.1, ZP_{—}00022726.1, EAA24844.1, ZP_{—}00029547.1, NP_{—}521484.1, NP_{—}240148.1, NP_{—}770852.1, AF345908_{—}2, NP_{—}777923.1, ZP_{—}00048879.1, NP_{—}579340.1, NP_{—}143455.1, NP_{—}126254.1, NP_{—}142573.1, NP_{—}613505.1, NP_{—}127112.1, NP_{—}712786.1, NP_{—}578214.1, NP_{—}069530.1, NP_{—}247526.1, AAA85593.1, NP_{—}212403.1, NP_{—}782258.1, ZP_{—}00058694.1, NP_{—}247137.1, NP_{—}219149.1, NP_{—}276946.1, NP_{—}614522.1, ZP_{—}00019288.1, CAD78330.1

The invention further relates to a genetically modified plant of the genus Tagetes, the genetic modification leading to an increasing or causing of the expression rate of at least one gene in comparison with the wild-type and being due to the regulation of the expression of this gene in the plant by the polynucleotide sequences according to the invention.

As mentioned above, “expression activity” is understood according to the invention as meaning the amounts of protein formed in a certain time by the promoter, that is the expression rate.

“Specific expression activity” is understood according to the invention as meaning the amount of protein per promoter formed in a certain time by the promoter.

In a “caused expression activity” or “caused expression rate” in relation to a gene in comparison with the wild-type, in comparison with the wild-type the formation of a protein is thus caused which was not present in the wild-type of the plant of the genus Tagetes.

For example, wild-type plants of the genus Tagetes have no ketolase gene. The regulation of the expression of the ketolase gene in the plant by the promoters according to the invention thus leads to a causing of the expression rate.

In an “increased expression activity” or “increased expression rate” in relation to a gene in comparison with the wild-type, in comparison with the wild-type the amount of protein formed in a certain time is thus increased in the plant of the genus Tagetes.

For example, wild-type plants of the genus Tagetes have a hydroxylase gene. The regulation of the expression of the hydroxylase gene in the plant by the promoters according to the invention thus leads to an increasing of the expression rate.

In a preferred embodiment of the genetically modified plants of the genus Tagetes according to the invention, the regulation of the expression of ketolase genes according to the invention is achieved by means of the promoters according to the invention, in that one or more nucleic acid constructs comprising at least one promoter according to the invention and, functionally linked, one or more ketolase genes to be expressed are inserted into the plant.

In a preferred embodiment one or more nucleic acid constructs comprising at least one promoter according to the invention and, functionally linked, one ketolase gene according to the invention and more genes to be expressed are inserted into the plant. The integration of the nucleic acid constructs into the plant of the genus Tagetes can in this case take place intrachromosomally or extrachromosomally.

Preferred promoters according to the invention and preferred genes to be expressed (effect genes) are described above.

By way of example, the production of the genetically modified plants of the genus Tagetes having an increased or caused expression rate of an effect gene is described below.

The transformation can be carried out individually or by means of multiple constructs in the combinations of genetic modifications.

The production of the transgenic plants is preferably carried out by transformation of the starting plants, using a nucleic acid construct which comprises at least one of the promoters according to the invention described above, which are functionally linked to an effect gene to be expressed and, if appropriate, to further regulation signals.

These nucleic acid constructs, in which the promoters according to the invention and effect genes are functionally linked, are also called expression cassettes below.

The expression cassettes can comprise further regulation signals, that is regulative nucleic acid sequences, which control the expression of the effect genes in the host cell. According to a preferred embodiment, an expression cassette comprises upstream, i.e. at the 5′-end of the coding sequence, at least one promoter according to the invention and downstream, i.e. at the 3′-end, a polyadenylation signal and, if appropriate, further regulatory elements which are operatively linked with the intermediate coding sequence of the effect gene for at least one of the genes described above.

An operative linkage is understood as meaning the sequential arrangement of promoter, coding sequence, terminator and, if appropriate, further regulative elements in such a way that each of the regulative elements can fulfill its function in the expression of the coding sequence as intended.

By way of example, the preferred nucleic acid constructs, expression cassettes and vectors for plants and processes for the production of transgenic plants, and the transgenic plants of the genus Tagetes themselves are described below.

The sequences which are preferred, but not restricted thereto, for the operative linkage are targeting sequences for the guaranteeing of the subcellular location in the apoplast, in the vacuoles, in plastids, in the mitochondrium, in the endoplasmatic reticulum (ER), in the cell nucleus, in elaioplasts or other compartments and translation enhancers such as the 5′-guiding sequence of the tobacco mosaic virus (Gallie et al., Nucl. Acids Res. 15 (1987), 8693-8711).

The production of an expression cassette is preferably carried out by fusion of at least one promoter according to the invention with at least one gene, preferably with one of the effect genes described above, and preferably a nucleic acid inserted between promoter and nucleic acid sequence, which codes for a plastid-specific transit peptide, and a polyadenylation signal according to customary recombination and cloning techniques, such as are described, for example, in T. Maniatis, E. F. Fritsch and J. Sambrook, Molecular Cloning: A Laboratory Manual, Cold Spring Harbor Laboratory, Cold Spring Harbor, N.Y. (1989) and also in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley-Interscience (1987).

The preferably inserted nucleic acids, encoding a plastidic transit peptide, guarantee location in plastids and in particular in chromoplasts.

Expression cassettes can also be used whose nucleic acid sequence codes for an effect gene-product fusion protein, a part of the fusion protein being a transit peptide which controls the translocation of the polypeptide. Specific transit peptides, which are removed enzymatically from the effect gene product part after translocation of the effect genes to the chromoplasts, are preferred for the chromoplasts.

The transit peptide which is derived from the plastidic Nicotiana tabacum transketolase or another transit peptide (e.g. the transit peptide of the small subunit of the Rubisco (rbcS) or of the ferredoxin NADP oxidoreductase and the isopentenyl pyrophosphate isomerase-2 or its functional equivalent) is particularly preferred.

Further examples of a plastidic transit peptide are the transit peptide of the plastidic isopentenyl pyrophosphate isomerase-2 (IPP-2) from Arabisopsis thaliana and the transit peptide of the small subunit of the ribulose bisphosphate carboxylase (rbcS) from pea (Guerineau, F, Woolston, S, Brooks, L, Mullineaux, P (1988) An expression cassette for targeting foreign proteins into the chloroplasts. Nucl. Acids Res. 16: 11380).

The nucleic acids according to the invention can be prepared synthetically or obtained naturally or comprise a mixture of synthetic and natural nucleic acid constituents, and consist of various heterologous gene sections of various organisms.

As described above, synthetic nucleotide sequences with codons which are preferred from plants are preferred. These codons preferred from plants can be determined from codons having the highest protein frequency, which are expressed in the most interesting plant species.

In the preparation of an expression cassette, various DNA fragments can be manipulated in order to obtain a nucleotide sequence which expediently reads in the correct direction and which is equipped with a correct reading frame. For the connection of the DNA fragments to one another, adapters or linkers can be added to the fragments.

Expediently, the promoter and the terminator regions can be provided in the transcription direction with a linker or polylinker which comprises one or more restriction sites for the insertion of this sequence. As a rule, the linker has 1 to 10, usually 1 to 8, preferably 2 to 6, restriction sites. In general, the linker within the regulatory areas has a size of less than 100 bp, frequently less than 60 bp, but at least 5 bp. The promoter can be either native or homologous, or foreign or heterologous for the host plant. The expression cassette preferably comprises, in the 5′-3′ transcription direction, the promoter, a coding nucleic acid sequence or a nucleic acid construct and a region for transcriptional termination. Various termination regions are mutually arbitrarily exchangeable.

Examples of a terminator are the 35S terminator (Guerineau et al. (1988) Nucl Acids Res. 16: 11380), the nos terminator (Depicker A, Stachel S, Dhaese P, Zambryski P, Goodman HM. Nopaline synthase: transcript mapping and DNA sequence. J Mol Appl Genet. 1982; 1 (6):561-73) or the ocs terminator (Gielen, J, de Beuckeleer, M, Seurinck, J, Debroek, H, de Greve, H, Lemmers, M, van Montagu, M, Schell, J (1984) The complete sequence of the TL-DNA of the Agrobacterium tumefaciens plasmid pTiAch5. EMBO J. 3: 835-846).

Furthermore, manipulations which make available suitable restriction cleavage sites or remove the superfluous DNA or restriction cleavage sites can be employed. Where insertions, deletions or substitutions such as, for example, transitions and transversions are possible, in vitro mutagenesis, “primer repair”, restriction or ligation can be used.

With suitable manipulations, such as, for example, restriction, “chewing-back” or filling of overhangs for “blunt ends”, complementary ends of the fragments can be made available for ligation.

Preferred polyadenylation signals are plant polyadenylation signals, preferably those which essentially correspond to T-DNA polyadenylation signals from Agrobacterium tumefaciens, in particular of gene 3 of the T-DNA (octopine synthase) of the Ti plasmid pTiACH5 (Gielen et al., EMBO J. 3 (1984), 835 ff) or functional equivalents.

The transposition of foreign genes into the genome of a plant is described as transformation.

To this end, methods known per se for the transformation and regeneration of plants from plant tissues or plant cells can be utilized for transient or stable transformation.

Suitable methods for the transformation of plants are protoplast transformation by polyethylene glycol-induced DNA uptake, the biolistic process using the gene gun—the “particle bombardment” method, electroporation, the incubation of dry embryos in DNA-containing solution, microinjection and gene transfer mediated by Agrobacterium, described above. Said processes are described, for example, in B. Jenes et al., Techniques for Gene Transfer, in: Transgenic Plants, Vol. 1, Engineering and Utilization, edited by S. D. Kung and R. Wu, Academic Press (1993), 128-143, and in Potrykus, Annu. Rev. Plant Physiol. Plant Molec. Biol. 42 (1991), 205-225.

Preferably, the construct to be expressed is cloned in a vector which is suitable for transforming Agrobacterium tumefaciens, for example pBin19 (Bevan et al., Nucl. Acids Res. 12 (1984), 8711) or particularly preferably pSUN2, pSUN3, pSUN4 or pSUN5 (WO 02/00900).

Agrobacteria transformed using an expression plasmid can be used in a known manner for the transformation of plants, e.g. by bathing wounded leaves or leaf pieces or cotyledons in an Agrobacteria solution and subsequently culturing in suitable media.

For the preferred production of genetically modified plants, also called transgenic plants below, the fused expression cassette is cloned in a vector, for example pBin19 or in particular pSUN5 and pSUN3, which is suitable to be transformed into Agrobacterium tumefaciens. Agrobacteria transformed using such a vector can then be used in a known manner for the transformation of plants, in particular of crop plants, by, for example, bathing wounded leaves or pieces of leaf or cotyledons in an Agrobacteria solution and subsequently culturing in suitable media.

The transformation of plants by Agrobacteria is known, inter alia, from F. F. White, Vectors for Gene Transfer in Higher Plants; in Transgenic Plants, Vol. 1, Engineering and Utilization, edited by S. D. Kung and R. Wu, Academic Press, 1993, pp. 15-38.

From the transformed cells of the wounded leaves or pieces of leaf or cotyledons, transgenic plants can be regenerated in a known manner which comprise one or more genes integrated into the expression cassette.

For the transformation of a host plant using one or more effect genes according to the invention, an expression cassette is incorporated into a recombinant vector as an insertion whose vector DNA comprises additional functional regulation signals, for example sequences for replication or integration. Suitable vectors are described, inter alia, in “Methods in Plant Molecular Biology and Biotechnology” (CRC Press), Chap. 6/7, pp. 71-119 (1993).

Using the recombination and cloning techniques cited above, the expression cassettes can be cloned into suitable vectors which make possible their proliferation, for example in E. coli. Suitable cloning vectors are, inter alia, pJIT117 (Guerineau et al. (1988) Nucl. Acids Res.16:11380), pBR332, pUC series, M13 mp series and pACYC184. Particularly suitable are binary vectors, which can replicate both in E. coli and in Agrobacteria.

The invention therefore furthermore relates to a genetically modified plant of the genus Tagetes, comprising a promoter according to the invention and, functionally linked, to a gene to be expressed, with the proviso that genes from plants of the genus Tagetes, which are expressed in wild-type plants of the genus Tagetes by the respective promoter, are excluded.

Preferred promoters and preferred effect genes according to the invention are described above.

Particularly preferably, effect genes are selected from the group consisting of nucleic acids encoding a ketolase, nucleic acids encoding a beta-hydroxylase, nucleic acids encoding a beta-cyclase, nucleic acids encoding an epsilon-cyclase, nucleic acids encoding an epoxidase, nucleic acids encoding an HMG-CoA reductase, nucleic acids encoding an (E)-4-hydroxy-3-methylbut-2-enyl diphosphate reductase, nucleic acids encoding a 1-deoxy-D-xylose-5-phosphate synthase, nucleic acids encoding a 1-deoxy-D-xylose-5-phosphate reductoisomerase, nucleic acids encoding an isopentenyl diphosphate delta-isomerase, nucleic acids encoding a geranyl diphosphate synthase, nucleic acids encoding a farnesyl diphosphate synthase, nucleic acids encoding a geranylgeranyl diphosphate synthase, nucleic acids encoding a phytoene synthase, nucleic acids encoding a phytoene desaturase, nucleic acids encoding a prephytoene synthase, nucleic acids encoding a zeta-carotene desaturase, nucleic acids encoding a crtlSO protein, nucleic acids encoding an FtsZ protein and nucleic acids encoding a MinD protein.

Preferred, genetically modified plants of the genus Tagetes are Marigold, Tagetes erecta, Tagetes patula, Tagetes lucida, Tagetes pringlei, Tagetes palmeri, Tagetes minuta or Tagetes campanulata.

By means of the promoters according to the invention, it is possible with the aid of the processes according to the invention described above to regulate, in the genetically modified plants of the genus Tagetes according to the invention described above, the metabolic pathways to specific biosynthetic products.

To this end, for example, metabolic pathways which lead to a specific biosynthetic product are strengthened by causing or increasing the transcription rate or expression rate of genes of this biosynthesis pathway by the increased amount of protein leading to an increased total activity of these proteins of the desired biosynthesis pathway and thus by an increased metabolic flow to the desired biosynthetic product.

Depending on the desired biosynthetic product, the transcription rate or expression rate of various genes must be increased or reduced. As a rule, it is advantageous to modify the transcription rate or expression rate of several genes, i.e. to increase the transcription rate or expression rate of a combination of genes and/or to reduce the transcription rate or expression rate of a combination of genes.

In the genetically modified plants according to the invention, at least one increased or caused expression rate of a gene is to be attributed to a promoter according to the invention.

Further, additionally modified, i.e. additionally increased or additionally reduced, expression rate of further genes in genetically modified plants can, but do not have to, be attributed to the promoters according to the invention.

The invention therefore relates to a process for the preparation of biosynthetic products by culturing genetically modified plants of the genus Tagetes according to the invention.

The invention relates in particular to a process for the production of carotenoids by culturing genetically modified plants of the genus Tagetes according to the invention, wherein the genes to be expressed are selected from the group consisting of nucleic acids encoding a ketolase, nucleic acids encoding a beta-hydroxylase, nucleic acids encoding a beta-cyclase, nucleic acids encoding an epsilon-cyclase, nucleic acids encoding an epoxidase, nucleic acids encoding an HMG-CoA reductase, nucleic acids encoding an (E)-4-hydroxy-3-methylbut-2-enyl-diphosphate reductase, nucleic acids encoding a 1-deoxy-D-xylose-5-phosphate synthase, nucleic acids encoding a 1-deoxy-D-xylose-5-phosphate reductoisomerase, nucleic acids encoding an isopentenyl diphosphate delta-isomerase, nucleic acids encoding a geranyl diphosphate synthase, nucleic acids encoding a farnesyl diphosphate synthase, nucleic acids encoding a geranylgeranyl diphosphate synthase, nucleic acids encoding a phytoene synthase, nucleic acids encoding a phytoene desaturase, nucleic acids encoding a prephytoene synthase, nucleic acids encoding a zeta-carotene desaturase, nucleic acids encoding a crtlSO protein, nucleic acids encoding an FtsZ protein and nucleic acids encoding a MinD protein.

The carotenoids are preferably selected from the group consisting of phytoene, phytofluene, lycopene, lutein, beta-carotin, zeaxanthin, astaxanthin, canthaxanthin, echinenone, 3-hydroxyechinenone, 3′-hydroxyechinenone, adonirubin, violaxanthin and adonixanthin.

In particular, the invention furthermore relates to a process for the production of ketocarotenoids by culturing genetically modified plants of the genus Tagetes according to the invention, wherein the genes to be expressed are selected from the group consisting of nucleic acids encoding a ketolase, nucleic acids encoding a beta-hydroxylase, nucleic acids encoding a beta-cyclase, nucleic acids encoding an epsilon-cyclase, nucleic acids encoding an epoxidase, nucleic acids encoding an HMG-CoA reductase, nucleic acids encoding an (E)-4-hydroxy-3-methylbut-2-enyl-diphosphate reductase, nucleic acids encoding a 1-deoxy-D-xylose-5-phosphate synthase, nucleic acids encoding a 1-deoxy-D-xylose-5-phosphate reductoisomerase, nucleic acids encoding an isopentenyl diphosphate delta-isomerase, nucleic acids encoding a geranyl diphosphate synthase, nucleic acids encoding a farnesyl diphosphate synthase, nucleic acids encoding a geranylgeranyl diphosphate synthase, nucleic acids encoding a phytoene synthase, nucleic acids encoding a phytoene desaturase, nucleic acids encoding a prephytoene synthase, nucleic acids encoding a zeta-carotene desaturase, nucleic acids encoding a crtlSO protein, nucleic acids encoding an FtsZ protein and nucleic acids encoding a MinD protein.

The ketocarotenoids are preferably selected from the group consisting of astaxanthin, canthaxanthin, echinenone, 3-hydroxyechinenone, 3′-hydroxyechinenone, adonirubin, violaxanthin and adonixanthin.

In the process according to the invention for the preparation of biosynthetic products, in particular carotenoids, preferably ketocarotenoids, a harvesting of the plants and an isolation of the biosynthetic products, in particular carotenoids, preferably ketocarotenoids, from the plants, preferably from the petals of the plants, is preferably added to the culturing step of the genetically modified plants.

The genetically modified plants of the genus Tagetes are grown in a manner known per se on nutrient media and appropriately harvested.

The isolation of ketocarotenoids from the harvested flower leaves is carried out, for example in a manner known per se, for example by drying and subsequent extraction and, if appropriate, further chemical or physical purification processes, such as, for example, precipitation methods, crystallography, thermal separation processes, such as rectifying processes or physical separation processes, such as, for example, chromatography. The isolation of ketocarotenoids from the flower leaves is carried out, for example, preferably by means of organic solvents such as acetone, hexane, heptane, ether or tert-methyl butyl ether.

Further isolation processes of ketocarotenoids, in particular from flower leaves, are described, for example, in Egger and Kleinig (Phytochemistry (1967) 6, 437-440) and Egger (Phytochemistry (1965) 4, 609-618).

A particularly preferred ketocarotenoid is astaxanthin.

The ketocarotenoids are obtained in the process according to the invention in flower leaves in the form of their mono- or diesters with fatty acids. Some proven fatty acids are, for example, myristic acid, palmitic acid, stearic acid, oleic acid, linolenic acid, and lauric acid (Kamata and Simpson (1987) Comp. Biochem. Physiol. Vol. 86B(3), 587-591).

Genetically modified plants or plant parts according to the invention consumable by humans and animals, such as, in particular, flower leaves having an increased content of biosynthetic products, in particular carotenoids, in particular ketocarotenoids, in particular astaxanthin, can also be used, for example, directly or after processing known per se, as foodstuffs or feedstuffs or as feed and food supplements.

Furthermore, the genetically modified plants can be used for the production of biosynthetic product-, in particular carotenoid-, in particular ketocarotenoid-, in particular astaxanthin-containing extracts and/or for the production of feed and food supplements, and of cosmetics and pharmaceuticals.

The genetically modified plants of the genus Tagetes have, in comparison with the wild-type, an increased content of the desired biosynthetic products, in particular carotenoids, in particular ketocarotenoids, in particular astaxanthin.

An increased content is in this case also understood as meaning a caused content of ketocarotenoids, or astaxanthin.

Another embodiment of the invention is a process for producing a fish diet comprising drying petals from lutein-depleted Tagetes which express a ketolase encoding polynucleotide sequence as mentioned above. For this process the above mentioned dried petals from Tagetes can be mixed with known ingredients and auxiliaries for fish diets. The petal containing diet can be formulated in known manners in order to achieve a good mixture or bioavailability of the ingredients.

The invention is illustrated by the examples which now follow, but is not restricted to these:

### EXPERIMENTAL SECTION

### Example 1

### Generation of Lutein-Depleted Tagetes Plants

The generation of a lutein-depleted Tagetes plant has been disclosed in U.S. Pat. No. 6,784,351 which is herewith completely incorporated by reference.

Naturally occurring Tagetes plants, which accumulate relatively high concentrations of beta-carotenoids while most of the alpha-carotenoids do not accumulate, are not known. Therefore, it was the task to develop a Tagetes plant which fulfills the requirements of i) being largely devoid of lutein and associated alpha-carotenoids in its flower petals and ii) being characterized by relatively high levels of total carotenoids. The process how Tagetes plants with lutein-depleted flowers were created is described in the U.S. Pat. No. 6,784,351. This patent describes in detail i) the EMS mutagenesis of Tagetes erecta “Scarletade” and “13819”, and ii) the HPLC screening procedure to identify certain abnormal carotenoid profiles in flowers of “Scarletade” and “13819”. The especially interesting mutant of Scarletade, 124-257, is described by its changed carotenoid profile in both petals and leaves.

### Example 2

### Breeding of 31360-2-09-08 and 31360-2-09

Tagetes erecta selection 124-257, described in U.S. Pat. No. 6,784,351, was found to have a low transformation rate using the identified tissue culture regeneration medium and Agrobacterium transformation technique. Using a standardized method, different plant selections can be transformed at different rates; therefore, to recover a target number of transformed plants, it can be expected that a selection having a low transformation rate would require use of a higher number of explants than a selection having a high transformation rate. A selection having a low transformation rate would require at least about 200 explants to recover about 1 transformed plant.

Instead of further optimizing the transformation protocol, a plant breeding back-cross technique well known to those skilled in the art was used to transfer the mutation resulting in the increased zeaxanthin to lutein ratio of selection 124-257 to a selection having a higher transformation rate. Several Tagetes erecta marigold plants were identified as having acceptable transformation rates, and from these Tagetes erecta marigold plant named 13819 was selected. Tagetes erecta 13819 is a proprietary breeding selection of PanAmerican Seed located at 622 Town Road, West Chicago, Ill. 60185.

In the backcross program, selection 124-257 was used as the female parent in a cross with a selection of 13819 as the male parent. The resulting population was identified as 11754, and from this population a plant identified as 11754-2F was selected based on its hybrid characteristics. Plant 11754-2F was selfed and from this population plant identified as 11754-2F-1 was selected based on carotenoid profile and plant habit. Plant 11754-2F-1 was selfed and from this population plant identified as 11754-2F-1-2 was selected based on carotenoid profile and plant habit. Plant 11754-2F-1-2 was used as male parent in a cross with 13819 as the female parent. The resulting population was identified as 31360, and from this population a plant identified as 31360-2 was selected based on carotenoid profile and plant habit. Plant 31360-2 was selfed and from the resulting population, plants 31360-2-08 and 31360-2-09 were selected based on carotenoid profile, total carotenoid concentration, and plant habit. Both selections were selfed and seed from the cross was used to test transformation rates, and the seedlings from both selections were found to have acceptable transformation rates. In addition, the resulting plants from the selfed 31360-2-08 plant were found to be uniform for carotenoid profile, carotenoid concentration, and plant habit. The resulting plants from the selfed 31360-2-09 plant were found to segregate for total carotenoid concentration and plant habit characteristics. From the selfed 31360-2-09 population, a plant identified as 31360-2-09-08 was selected based on carotenoid profile, total carotenoid concentration, and plant habit. The selfed population from the 31360-2-09-08 plant was found to be uniform for carotenoid profile, carotenoid concentration, and to have an acceptable transformation rate.

### Example 3

### Synthesis of the Coding Sequence of the Beta-Carotene Ketolase from Chlorella zofingiensis

The accession number B1934406 from the public sequence (“Chlorella zofingiensis beta-carotene ketolase/oxygenase (bkt/crto) gene, complete cds”) was used to identify the coding sequence of the beta-carotene ketolase from Chlorella zofingiensis. To isolate the DNA fragment described by SEQ ID NO:1, the coding sequence of the beta-carotene ketolase from Chlorella (position 209-1147; =SEQ ID NO:2), fused to coding sequence of the transit peptide of the pisum sativum Rubisco smale subunit (rbcS) gene (position 38-205), was synthesized by the firm Entelechon GmbH (Regensburg, Germany).

### Example 4

### Cloning of the Coding Sequence of the Beta-Carotene Ketolase from Scenedesmus vacuolatus Strain 211-8b from the Culture Collection of the Universtiy of Goettingen (SAG)

To isolate the DNA fragment described by SEQ ID NO: 5, Scenedesmus vacuolatus SAG211-8b was grown as for 14 days under low light conditions in basal media with peptone, as recommended by the culture collection of the University of Goettingen (SAG). RNA was isolated from tissue of Scenedesmus vacuolatus according to published methods (e.g. Maniatis T, Fritsch E F, and Sambrook J Molecular Cloning: A Laboratory Manual, Cold Spring Harbor Laboratory, Cold Spring Harbor (NY), (1989); Qiagen, RNeasy Mini Handbook 06/2001). The isolated RNA was employed as matrix for cDNA synthesis according to published methods (e.g. Maniatis T, Fritsch E F, and Sambrook J Molecular Cloning: A Laboratory Manual, Cold Spring Harbor Laboratory, Cold Spring Harbor (NY), (1989)). The resulting cDNA was employed as matrix DNA for a polymerase chain reaction (PCR) mediated amplification using the oligonucleotide primers SEQ ID NO: 3 and SEQ ID NO: 4. Alternatively, the fragment described by SEQ ID NO: 5 can be generated by in vitro synthesis.

| | | |
|---|---|---|
| SEQ ID NO 3: | SVK-10 | gcgcatatggctcccaggcggcaa |
| SEQ ID NO 4: | SVK-11 | CGGTCGACTTACTCCACTACTGCTCC |

SEQ ID NO 5: Scenesdesmus vacuolatus beta-carotene coding sequence
Position 1 to 24 Primer binding region for primer SVK-10
Position 985 to 1010 Primer binding region for primer SVK-11
Position 7 to 1002 region coding for beta-carotene ketoalse from Scenesdesmus vacuolatus (═SEQ ID NO:6)
PCR amplification was carried out as follows:
100 ng cDNA
1×PCR buffer

### 2.5 mM MgCl2,

200 μM each of dATP, dCTP, dGTP und dTTP
10 μmol of each oligonucleotide primers
2.5 Units Pfu DNA Polymerase (Stratagene) in a final volume of 50 μl

The following temperature program is employed for the various amplifications (BIORAD Thermocycler).

### 1. 95° C. for 5 min

2. 54° C. for 1 min, followed by 72° C. for 5 min and 95° C. for 30 sec. Repeated 25 times.
3. 54° C. for 1 min, followed by 72° C. for 10 min.

### 4. Storage at 4° C.

The resulting PCR-products are cloned in a standard cloning vector and then reisolated by digestion with the suitable restriction endonucleases and cloned into a vector (predigested with the same enzymes) downstream of the promoter (e.g. CHRC promoter) and upstream of the terminator region (e.g. LB3 terminator) in operable linkage. This expression cassette is used to create vectors for stable transformation. This resulting vector is used for stable transformation of Tagetes, for example in germplasm described in example 1 and according to the procedure described in example 2. Biochemical analysis of petal tissue is performed as described in example 3.

### Example 5

### Vector Construction for Overexpression Experiments with the Ketolase from Chlorella zofingiensis and Scenedesmus vacuolatus SAG211-8b in Tagetes erecta

Vectors used for expression of the Scenedesmus vacuolatus & Chlorella zofingiensis beta-carotene ketolase genes in plants (overexpression) are designed to overexpress the ketolase under control of suitable promoters and are of two general types, biolistic and binary, depending on the plant transformation method to be used.

### For Biolistic Transformation (Biolistic Vectors), the Requirements are as Follows:

1. a backbone with a bacterial selectable marker (typically, an antibiotic resistance gene) and origin of replication functional in Escherichia coli (E. coli; e.g., ColE1), and
2. a plant-specific portion consisting of:
   a. a gene expression cassette consisting of a promoter (e.g., ZmUBlint MOD), the gene of interest (typically, a full-length cDNA) and a transcriptional terminator (e.g., Agrobacterium tumefaciens nos terminator);
   b. a plant selectable marker cassette, consisting of a suitable promoter, selectable marker gene (e.g., pat; D-amino acid oxidase; daol) and transcriptional terminator (eg. nos terminator).

Vectors designed for transformation by Agrobacterium tumefaciens (A. tumefaciens; binary vectors) consist of:
1. a backbone with a bacterial selectable marker functional in both E. coli and A. tumefaciens (e.g., spectinomycin resistance mediated by the aadA gene) and two origins of replication, functional in each of aforementioned bacterial hosts, plus the A. tumefaciens virG gene;
2. a plant-specific portion as described for biolistic vectors above, except in this instance this portion is flanked by A. tumefaciens right and left border sequences which mediate transfer of the DNA flanked by these two sequences to the plant.
   Base Vector used for Cloning of Overexpression Constructs
   SEQ ID NO. 7: VC-LLL544-lqcz (binary vector backbone)
   Position 1 to 146 Right border
   Position 320 to 1111 Adenyltransferase [aadA] gene coding region
   Position 1560 to 2241 ColE1 E. coli origin of replication
   Position 2615 to 2809 pVS1 origin (complementary)
   Position 2413 to 5682 pVS1 replicon (complementary)
   Position 5691 to 5905 Left border
   Vector Construction for Overexpression Experiments with Chlorella zofingiensis Beta-Carotene Ketolase
SEQ ID NO. 8: T-DNA of binary vector with expression cassette BnPAPX:CZ BKT:CAT term (VC-SMS309)
   Position 3 to 290 Nos (nopaline synthase) gene promoter
   Position 303 to 854 Phosphinothricin Acetyltransferase synthetic gene/CDS
   Position 924 to 1176 Nos (nopaline synthase) gene terminator
   Position 1367 to 1598 Solanum tuberosum Cathepsin D Inhibitor (CAT) terminator (complementary)
   Position 1614 to 2552 Chlorella zofingiensis beta-carotene ketolase gene coding sequence (complementary)
   Position 2556 to 2723 Pisum sativum Rubisco small subunit (RbcS) transit peptide coding sequence (complementary)
   Position 2761 to 4798 Arabidopsis thaliana Triose phosphate translocator (TPT) promoter (complementary)
   Vector Construction for Overexpression Experiments with Scenedesmus vacuolatus beta-carotene ketolase
SEQ ID NO. 9: T-DNA of binary vector with expression cassette BnPAPX:SV211 BKT:CAT term (VC-SMS330)
   Position 3 to 290 Nos (nopaline synthase) gene promoter
   Position 303 to 854 Phosphinothricin Acetyltransferase synthetic gene/CDS
   Position 924 to 1176 Nos (nopaline synthase) gene terminator
   Position 1367 to 1598 Solanum tuberosum Cathepsin D Inhibitor (CAT) terminator (complementary)
   Position 1660 to 2655 Scenedesmus vacuolatus SAG 211-8b beta-carotene ketolase gene coding sequence (complementary)
   Position 2659 to 2826 Pisum sativum Rubisco small subunit (RbcS) transit peptide coding sequence (complementary)
   Position 2860 to 3890 Brassica napus Plastid-Associated protein X (PAPX) promoter (complementary)

### Example 8

### Transformation of Tagetes 31360-2-08

Seeds of Tagetes erecta line 31360-2-08 were disinfected with 2% NaOCl solution for 10 minutes followed by three washes with autoclaved distilled water. Afterwards seeds were dried and can be stored under aseptic conditions at room temperature for a period of up to two weeks before in vitro germination. Germination occurred on solidified MS medium (Murashige, T., and Skoog, F., A revised medium for rapid growth and bioassays with tobacco tissue cultures. Physiol. Plant. 15, 473-497, 1962) in a 16/8 h light/darkness photoperiod for 1-3 weeks. Cotyledonary segments were prepared and used as primary target material for transformation. These segments were inoculated for 20 minutes in liquid MS medium containing Agrobacterium tumefaciens strain EHAL05 [carrying the vector VC-SMS330 or VC-SMS309] cells at an OD₆₀₀ of 0.1. The construct VC-SMS330 or VC-SMS309 contained the gene pat and allows therefore phosphinothricin (PPT) and/or BASTA selection. Explants are co-cultured for a period of 6 days on MS medium (pH 5.8) solidified with 0.8% agar and supplemented with 1 mg/l 3-indole-3-acetic acid (IAA), 3 mg/l indole-3-butyric acid (IBA), 500 mg/12-(N-morpholino) ethanesulfonic acid (MES) and 2% sucrose. Cultivation occurred under controlled conditions at 21° C., 35-40 μmol m⁻² s⁻¹ white light intensity and 16/8 light/darkness rhythm. Shoots were induced on cotyledon explants on fresh MS medium, adjusted to pH 5.5, as described before supplemented with 500 mg/l Timentin, 1 mg/l PPT, 5 mg/l silver nitrate (AgNO₃). Second to fourth subcultures were done onto fresh MS medium following the formulation described above at pH 5.8 and 15 days subculture period. The newly formed shoot buds were transferred to a new medium to promote shoot regeneration. The shoot regeneration medium follows the formulation of MS supplemented with 0.7% agar, 250 mg/l Timentin, 1 mg/l PPT, 5 mg/l AgNO₃, 1 mg/l IAA, 3 mg/16-Benzylaminopurine (BAP), 500 mg/l MES and 2% sucrose and was adjusted to pH 5.8. Three subcultures were promoted in a 15 days subculture period. Regenerated shoots were then transferred onto elongation medium (MS) supplemented with 0.7% agar, 250 mg/l Timentin, 1 mg/l PPT, 5 mg/l AgNO₃, 0.5 mg/l IAA, 0.5 mg/l gibberellic acid (GA₃), 500 mg/l MES, 2% sucrose, pH 5.8. Three subcultures were performed, each for 15 days. Well elongated shoots (1.5-3.5 cm in length) with well expanded leaves were transferred onto rooting MS medium solidified with 0.7% agar and supplemented with 250 mg/l Timentin, 1 mg/l PPT, 0.5 mg/l IBA, 500 mg/l MES, 2% sucrose and adjusted to pH 5.8. Leaf material from rooted plants was analyzed by qPCR for the selection marker gene in order to confirm transgenicity and to determine the copy number of the construct integrated into the genome. After four weeks the well rooted transgenic shoots were transferred to ex vitro-conditions at the greenhouse. Hardening of plants in soil could be achieved with inverted funnels. They prevented dehydration of the plantlets. Afterwards plants were transferred into bigger pots with soil to promote growth and development until flowering under greenhouse conditions.

### Example 9

### Enzymatic Lipase-Catalysed Hydrolysis of Carotenoid Esters from Plant Extracts and Identification of Individual Carotenoids

### a) General Working Procedure

Homogenized plant material from Tagetes, e.g. material of petals (30-100 mg fresh weight), was extracted with 100% acetone (three times with 500 μl; each time with vigorous shaking for about 15 minutes). The combined solvent volumes were evaporated. Then, carotenoids were resuspended in 495 μl acetone and 4,95 ml potassium phosphate buffer (100 mM, pH 7.4) was added and intensely mixed. 17 mg Bile-salts (Sigma; the Bile-salts used are 1:1 mixtures of cholate and desoxycholate.) and 149 μl of a NaCl/CaCl₂-solution (3M NaCl and 75 mM CaCl₂) were added. The suspension was incubated for 30 minutes at 37° C. To hydrolyse the carotenoid esters enzymatically, 595 μl of a lipase solution (50 mg/ml lipase type 7 of Candida rugosa (Sigma) were added and incubated under constant shaking at 37° C. After 21 hours, again 595 μl lipase solution were added, followed by an incubation at 37° C. for at least 5 hours. After the incubation, about 700 mg Na₂SO₄ (free of water) were added to the solution and solved. 1800 μl of petrolether was given to the solution and the carotenoids were extracted into the organic phase via vigorous and intensive mixing. The petrolether phase was taken off and fresh petrolether was added again, followed by vigorous mixing. This procedure was repeated till the organic phase stayed colorfree. The collected petrolether phases were combined and the petrolether was then evaporated in the vacuum. Released and therefore free carotenoids were solved in 100-120 μl acetone. Using HPLC and a C30 reverse phase-column, carotenoids were identified based on their retention times and UV-VIS-spectra (in comparison to commercially available standard compounds).

### b) Method for Low Concentrations of Carotenoid Esters in Plant Material

Alternatively to the procedure described above, hydrolysis of carotenoid esters by lipase from Candida rugosa can be performed after separation of the extract on TLC plates (TLC=thin layer chromatography). 50-100 mg fresh plant material was extracted three times with 750 μl acetone. The combined organic supernatants were evaporated in the vacuum (elevated temperatures of 40-50° C. can be tolerated). Extracted carotenoids were solved in 300 μl petrolether:acetone (ratio 5:1) and well mixed. Solid particles were pelleted by centrifugation (15.000 g) for 1-2 minutes. The upper phase was transferred to a new reaction tube. The remainder was re-extracted with 200 μl petrolether:acetone (ratio 5:1) and particles were again removed by centrifugation. Supernatants were combined and the solvents were evaporated. The remainder was resuspended in 30 μl petrolether:acetone (ratio 5:1) and spotted onto a TLC plate (thin layer chromatography plate; Silica-Gel 60, Merck, Germany). In case more than one spotting was required for preparative-analytical purposes, several aliquots each of 50-100 mg fresh weight should be prepared as described above.

The TLC plates were developed in petrolether:acetone (ratio 5:1). Bands of carotenoids could be identified visually due to their colors. Individual carotenoid bands were scraped out and sometimes pooled for preparative analysis. Carotenoids were dissolved from the Silica material with acetone; the solvent was later evaporated in the vacuum. The remainder was solved in 495 μl acetone for the hydrolysis of carotenoid esters, 17 mg Bile-salts (Sigma) and 4,95 ml 0.1M potassium phosphate buffer (pH 7.4) and 149 μl of (3M NaCl, 75 mM CaCl₂) were added. After mixing all components well, the mixture was equilibrated for 30 minutes at 37° C. Then, 595 μl lipase from Candida rugosa (Sigma, stock solution of 50 mg/ml in 5 mM CaCl₂) was added; incubation with lipase was done over night at 37° C. under constant shaking. After 21 hours of incubation, an equal amount of lipase was added; the incubation proceeded for at least an additional 5 hours under constant shaking. Afterwards, 700 mg Na₂SO₄ (free of water) were given to the solution and mixed well with 1800 μl added petrolether for 1 minute; the mixture was centrifuged at 3500 g for 5 minutes. The upper phase was transferred into a new reaction vial, and the extraction of carotenoids into the petrolether phase was repeated several times. The extraction was finished when the upper phase remained colorless after mixing. The pooled petrolether phases were evaporated in the vacuum (temperatures of 40-50° C. were acceptable). The remainder was solved in 120 μl acetone, eventually with short sonication. The solved carotenoids were analyzed via HPLC using a C30-column and quantified using reference compounds as standards.

### KOH Saponification of Xanthophylls

10-20 mg fresh material of Tagetes petals were homogenized (via mortar and pestle in liquid nitrogen). The homogenous material was extracted with acetone, usually three times with 500 ul acetone till the supernatant is colorless. If needed, material was shaken after each extraction. All supernatants were combined and evaporated to dryness using a speedvac concentrator. The pellet was dissolved in 180 μl of acetone and eventually briefly sonicated. For saponification, 20 μl of 10% KOH (in methanol) was added and incubated for 30 minutes under constant shaking (1000 rpm) in the dark at room temperature. The reaction was stopped by the addition of 20-30 μl 1 M HCl (till neutral pH value was reached). Samples were centrifuged for 10 min at 13.000 rpm to pellet debris and analyzed by HPLC.

### Example 10

### HPLC Analysis of Free Carotenoids

The analysis of samples prepared according to the procedure described above was done under the following conditions:

HPLC conditions:
HPLC column: Prontosil C30, 250×4.6 mm, (Bischoff, Leonberg, Germany)
Flow rate: 1.0 ml/min
Eluents: Solvent A—100% methanol
Solvent B—80% methanol, 0.2% ammoniumacetate
Solvent C—100% t-butyl-methylether

### Detection: 300-530 nm

### Gradienten Profile:

| Time (min) | Flow rate | % Solvent A | % Solvent B | % Solvent C |
|---|---|---|---|---|
| 1.00 | 1.0 | 95.0 | 5.0 | 0 |
| 12.00 | 1.0 | 95.0 | 5.0 | 0 |
| 12.10 | 1.0 | 80.0 | 5.0 | 15.0 |
| 22.00 | 1.0 | 76.0 | 5.0 | 19.0 |
| 22.10 | 1.0 | 66.5 | 5.0 | 28.5 |
| 38.00 | 1.0 | 15.0 | 5.0 | 80.0 |
| 45.00 | 1.0 | 95.0 | 5.0 | 0 |
| 46.0 | 1.0 | 95.0 | 5.0 | 0 |

Some typical retention times for carotenoids are:

violaxanthin at about 11.7 min, astaxanthin at about 17.7 min, adonixanthin at about 19 min, adonirubin at about 19.9 min, zeaxanthin at about 21 min.

After transformation of Tagetes explants of 31360-2-08 with the binary vector VC-SMS309, several transgenic plants were obtained which were named MS309-71, 197 & 137. After transformation of Tagetes explants of 31360-2-08 with the binary vector VC-SMS330, several transgenic plants were obtained which were named MS309-5, 15, 29 & 97. These plants were analyzed for individual yellow carotenoids, the natural occurring and endogenous xanthophylls, and the newly formed ketocarotenoids, especially for astaxanthin, canthaxanthin, echinenone, 3′-hydroxechinenone, 3-hydroxechinenone, phoenicoxanthin (=adonirubin) and adonixanthin.

TABLE 1
Individual carotenoids in petals of transgenic Tagetes MS309

| | ng/mg | | | | | | |
|---|---|---|---|---|---|---|---|
| | DW | | | % | | | |
| Plant | total caros | Ketos | Asta | A + P | A + P + C | bC + Cryp + Zea | Epoxides |
| MS309-71 | 13225 | 0.2 | 0.05 | 0.07 | 0.07 | 57.3 | 41.6 |
| MS309-107 | 12656 | 0.4 | 0.17 | 0.2 | 0.02 | 52.3 | 46.6 |
| MS309-137 | 12090 | 0.1 | 0.04 | 0.05 | 0.06 | 48.7 | 50.3 |

Listed values represent individual carotenoids in percent of total carotenoids, extracted and analyzed as described. The carotenoid extract was prepared from fully opened Tagets flowers. Value refer to fresh weight.

Legend for table 1 and following:

“total caros”: total amounts of all carotenoids extracted from Tagets petals
“Ketos”: sum of all ketocarotenoids extracted from Tagetes petals (canthaxanthin, phoenicoxanthin, Aastaxanthin, adonixanthin, echinenone, 3′-und 3-hydroxyechinenone).
“Asta”: designation for the ketocarotenoid astaxanthin
“Adoni”: designation for the ketocarotenoid adonixanthin
“P” and “Phoenico”: designation for the ketocarotenoid phoenicoxanthin, also known as adonirubin
“C” and “Cantha”: designation for the ketocarotenoid canthaxanthin
“bC”: designation for beta-carotene
“Cryp”: designation for beta-cryptoxanthin
“Zea”: designation for zeaxanthin
“Cantha” designation for canthaxanthin
“3”-Hydroxy” and “HO-echi”: designation for 3′-hydroxyechinenone
“b-Crypto” bezeichnet beta-Cryptoxanthin
“Epoxides”: designation for the combined concentration of the carotenoid epoxides violaxanthin, antheraxanthin and neoxanthin
“DW” stands for dry weight

TABLE 2
Individual carotenoids in petals of transgenic Tagetes MS330

| | ng/mg | | | | | | | | |
|---|---|---|---|---|---|---|---|---|---|
| | DW | | | % | | | | | |
| | total | | | | | | | | |
| Plant | caros | Ketos | Asta | A + P | A + P + C | bC | Cryp | Zea | Epoxides |
| MS330-5 | 13410 | 20.7% | 16.7% | 18.8% | 20.5% | 67.9% | 0.3% | 1.2% | 1.0% |
| MS330-15 | 10459 | 20.3% | 14.6% | 17.6% | 20.0% | 66.9% | 0.2% | 1.4% | 1.6% |
| MS330-29 | 9961 | 35.4% | 27.3% | 32.1% | 35.0% | 54.6% | 0.2% | 1.6% | 1.6% |
| MS330-97 | 8525 | 22.8% | 17.0% | 20.1% | 22.4% | 67.4% | 0.2% | 1.5% | 1.4% |
| MS330-103 | 14813 | 21.2% | 15% | 18.5% | 21.0% | 71.6% | 0.1% | 1.1% | 0.6% |
| MS330-107 | 11395 | 20.1% | 14% | 17.4% | 19.8% | 68.5% | 0.1% | 1.1% | 0.8% |
| MS330-126 | 10856 | 23.2% | 16% | 20.1% | 22.8% | 65.7% | 0.2% | 0.9% | 1.2% |
| MS330-131 | 9064 | 19.5% | 15% | 17.5% | 19.4% | 69.9% | 0.2% | 1.1% | 2.0% |
| MS330-137 | 11986 | 27.2% | 21% | 24.5% | 27.1% | 65.9% | 0.2% | 0.9% | 0.9% |
| SMS330-2 | 14012 | 20.8% | 16% | 18.6% | 20.6% | 64.8% | 0.2% | 0.9% | 2.6% |
| SMS330-20 | 13113 | 27.0% | 20% | 24.3% | 26.8% | 60.4% | 0.1% | 0.8% | 1.9% |
| SMS330-72 | 15119 | 21.0% | 16% | 18.4% | 20.7% | 69% | 0.2% | 1.0% | 1.6% |
| SMS330-92 | 16085 | 18.3% | 12% | 15.3% | 18.1% | 73.4% | 0.1% | 0.9% | 1.5% |
| SMS330-115 | 15460 | 21.1% | 16% | 18.3% | 20.8% | 68.7% | 0.1% | 1.0% | 1.0% |
| SMS330-133 | 8969 | 25.6% | 19% | 22.9% | 25.3% | 63.6% | 0.2% | 1.1% | 2.7% |
| SMS330-18 | 13604 | 16.0% | 9% | 13.1% | 15.9% | 59.5% | 0.3% | 4.3% | 14.5% |
| SMS330-68 | 12528 | 23.0% | 13% | 18.7% | 22.7% | 65.9% | 0.1% | 1.6% | 1.7% |
| SMS330-74 | 11707 | 20.3% | 16% | 18.5% | 20.1% | 65.3% | 0.2% | 0.9% | 3.3% |
| SMS330-87 | 19625 | 2.4% | 2% | 2.1% | 2.3% | 66.7% | 0.5% | 9% | 19.3% |
| SMS330-98 | 15402 | 23.6% | 17% | 20.8% | 23.3% | 68.1% | 0.1% | 0.7% | 1.1% |
| SMS330-112 | 13619 | 7.0% | 5% | 5.7% | 7.0% | 60% | 0.4% | 13% | 13.6% |

Listed values represent individual carotenoids in percent of total carotenoids, extracted and analyzed as described. The carotenoid extract was prepared from fully opened Tagetes flowers. Value refer to fresh weight.

TABLE 3
Individual ketocarotenoids in petals of transgenic Tagetes MS330

| | | | | | HO- | |
|---|---|---|---|---|---|---|
| Plant | Asta | Adoni | Phoenico | Cantha | echi | Ketos |
| MS330-5 | 80.6% | 0.5% | 10.5% | 7.8% | 0.6% | 20.7% |
| MS330-15 | 71.7% | 0.6% | 14.6% | 12.0% | 1.1% | 20.3% |
| MS330-29 | 76.9% | 0.4% | 13.7% | 8.1% | 0.9% | 35.4% |
| MS330-97 | 74.8% | 0.3% | 13.7% | 10.2% | 1.1% | 22.8% |
| MS330-103 | 72.1% | 0.5% | 15.2% | 11.5% | 0.7% | 21.2% |
| MS330-107 | 69.6% | 0.5% | 16.6% | 12.1% | 1.1% | 20.1% |
| MS330-126 | 69.9% | 0.5% | 16.6% | 12.0% | 1.0% | 23.2% |
| MS330-131 | 78.5% | 0.4% | 11.0% | 9.6% | 0.5% | 19.5% |
| MS330-137 | 75.8% | 0.4% | 14.0% | 9.6% | 0.2% | 27.2% |
| SMS330-2 | 76.0% | 0.5% | 13.5% | 9.4% | 0.6% | 20.8% |
| SMS330-20 | 75.2% | 0.3% | 14.6% | 9.3% | 0.6% | 27.0% |
| SMS330-72 | 74.6% | 0.6% | 13.0% | 10.9% | 0.8% | 21.0% |
| SMS330-92 | 66.9% | 0.5% | 16.7% | 14.9% | 1.0% | 18.3% |
| SMS330-115 | 74.8% | 0.5% | 11.9% | 11.9% | 0.9% | 21.1% |
| SMS330-133 | 75.8% | 0.6% | 13.8% | 9.0% | 0.8% | 25.6% |
| SMS330-18 | 54.8% | 0.2% | 27.4% | 16.9% | 0.7% | 16.0% |
| SMS330-68 | 58.1% | 0.2% | 23.3% | 17.1% | 1.2% | 23.0% |
| SMS330-74 | 77.0% | 0.4% | 14.0% | 7.8% | 0.8% | 20.3% |
| SMS330-87 | 75.8% | 1.4% | 10.6% | 10.4% | 1.8% | 2.4% |
| SMS330-98 | 73.1% | 0.3% | 15.1% | 10.7% | 0.7% | 23.6% |
| SMS330-112 | 64.2% | 0.2% | 17.4% | 17.6% | 0.5% | 7.0% |

1. A ketolase comprising
the polypeptide sequence of SEQ ID NO:6,
a sequence derived from the sequence of SEQ ID NO:6 by substitution, insertion or deletion of one or more amino acids, which has an identity of at least 60% with the sequence of SEQ. ID. NO: 6 or
a functionally equivalent fragment of the sequences mentioned above.

2. A polynucleotide sequence encoding the ketolase according to claim 1.

3. The polynucleotide sequence according to claim 2 comprising the sequence of SEQ ID NO:5.

4. A process for the production of keto-carotenoids in lutein-depleted plants of the genus Tagetes a comprising utilizing the polynucleotide sequence according to claim 2.

5. The process according to claim 4 wherein the polynucleotide sequence encoding a ketolase is under the transcriptional control of a plastid-lipid associated protein-promoter (PAP-promoter).

6. The process according to claim 5, wherein the PAP-promoter comprises the sequence of SEQ ID NO:10 or SEQ ID NO:11.

7. The process according to claim 4, wherein expression takes place specifically in petals.

8. The process according to claim 4, wherein the keto-carotenoid is astaxanthin.

9. The process according to claim 4, further comprising culturing a lutein-depleted Tagetes and harvesting the petals of the Tagetes.

10. A process for producing a fish diet comprising drying petals from a lutein-depleted Tagetes which expresses the polynucleotide sequence according to claim 2.

11. A process for the production of keto-carotenoids in lutein-depleted plants of the genus Tagetes comprising transforming a lutein-depleted plant cell of the genus Tagetes with the polynucleotide sequence of claim 2 and obtaining a plant which expresses the polynucleotide.

12. A transgenic plant or plant cell produced by the method of claim 11.
